(19) United States
(12) Patent Application Publication
(10) Pub. No.: US 2025/0326726 A1
(43) Pub. Date: Oct. 23, 2025

(54) FERROPTOSIS INHIBITORS-PHENOXAZINE ACETAMIDES

(71) Applicant: SIRONAX (BEIJING) CO., LTD., Beijing (CN)

(72) Inventors: Jianguang Han, Beijing (CN); Zhiyuan Zhang, Beijing (CN); Yanping Xu, Beijing (CN); Yimin Jiang, Beijing (CN)

(21) Appl. No.: 18/868,281

(22) PCT Filed: Apr. 3, 2023
(86) PCT No.: PCT/CN23/85826

§ 371 Date: Nov. 22, 2024

(51) Int. Cl. C07D 265/38; A61K 31/538; A61K 31/551; C07D 413/12; C07D 453/02; C07D 487/08

(52) U.S. Cl. CPC C07D 265/38; A61K 31/538; A61K 31/551; C07D 413/12; C07D 453/02; C07D 487/08

(57) ABSTRACT

Provided herein are compounds that inhibit ferroptosis activity, or modulate or inhibit a disease associated with ferroptosis dysregulation, such as neuropathy, ischemia reperfusion injury, acute kidney failure and cancer, including corresponding sulfonamides, and pharmaceutically acceptable salts, hydrates and stereoisomers thereof. The compounds are employed in pharmaceutical compositions, and methods of making and use, including treating a person in need thereof with an effective amount of the compound or composition, and detecting a resultant improvement in the person's health or condition.

### INTRODUCTION

Ferroptosis is a type of programmed cell death dependent on iron and characterized by the accumulation of lipid peroxides, and is genetically and biochemically distinct from other forms of regulated cell death such as apoptosis, autophagy and necrosis. Dysregulated ferroptosis has been implicated in a number of diseases, including neuropathy, ischemia reperfusion injury, acute kidney failure and cancer.

### SUMMARY OF THE INVENTION

The invention provides compounds that modulate or inhibit ferroptosis activity, or modulate or inhibit a disease associated with ferroptosis dysregulation, such as neuropathy, ischemia reperfusion injury, acute kidney failure and cancer, and prodrugs thereof, which are hydrolyzed, typically in the gut or blood, to yield the corresponding compounds/inhibitors.

In an aspect the invention provides a compound of formula I, or a salt, hydrate or stereoisomer thereof:

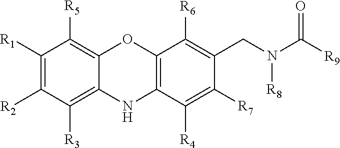

wherein:
R1-R8 are independently H, optionally substituted heteroatom or optionally substituted hydrocarbyl; and
R9 is optionally substituted alkyl or optionally substituted, optionally heterocyclic.

In embodiments:
R1-R7 are independently H, optionally substituted heteroatom or optionally substituted hydrocarbyl;
R1-R7 are independently H, halide, optionally substituted OH or NH2, or optionally substituted alkyl;
R1-R7 are independently H or optionally F-substituted lower alkyl;
R8 is H, optionally substituted heteroatom or optionally substituted hydrocarbyl;
R8 is H, optionally substituted OH or optionally substituted alkyl;
R8 is H or OH;
R9 is optionally substituted alkyl or optionally substituted, optionally hetero cyclic;
R9 is substituted methyl;
R9 is methyl substituted with optionally substituted optionally heterocyclic;
or 5 of R3-R7 are H; or
any combination of the foregoing substituents.

In an aspect the invention provides a compound disclosed herein, or a salt, hydrate or stereoisomer thereof:

In an aspect the invention provides a pharmaceutical composition comprising a therapeutically effective amount of a compound for formula I (supra) in predetermined, unit dosage form and one or more pharmaceutically acceptable excipients.

In an aspect the invention provides use of a compound or composition disclosed herein in the manufacture of a medicament to inhibit ferroptosis activity, or modulate or inhibit a disease associated with ferroptosis dysregulation, such as neuropathy, ischemia reperfusion injury, acute kidney failure and cancer in a person in need thereof.

In an aspect the invention provides a compound or composition disclosed herein to inhibit ferroptosis activity, or modulate or inhibit a disease associated with ferroptosis dysregulation, such as neuropathy, ischemia reperfusion injury, acute kidney failure and cancer, in a person in need thereof, or in the manufacture of a medicament thereof in a person in need thereof.

In an aspect the invention provides a method of using a compound or composition disclosed herein to inhibit ferroptosis activity, or modulate or inhibit a disease associated with ferroptosis dysregulation, such as neuropathy, ischemia reperfusion injury, acute kidney failure and cancer, in a person in need thereof, and optionally detecting a resultant improvement in the person's health or condition.

The invention encompasses all combination of the particular embodiments recited herein, as if each combination had been laboriously recited.

### DESCRIPTION OF PARTICULAR EMBODIMENTS OF THE INVENTION

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

The term “alkyl” refers to a hydrocarbon group selected from linear and branched saturated hydrocarbon groups of 1-18, or 1-12, or 1-6 carbon atoms. Examples of the alkyl group include methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of the alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

Lower alkyl means 1-8, preferably 1-6, more preferably 1-4 carbon atoms; lower alkenyl or alkynyl means 2-8, 2-6 or 2-4 carbon atoms.

The term “alkenyl” refers to a hydrocarbon group selected from linear and branched hydrocarbon groups comprising at least one C═C double bond and of 2-18, or 2-12, or 2-6 carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups.

The term “alkynyl” refers to a hydrocarbon group selected from linear and branched hydrocarbon group, comprising at least one C≡C triple bond and of 2-18, or 2-12, or 2-6 carbon atoms. Examples of the alkynyl group include ethynyl, 1-propynyl, 2-propynyl (propargyl), 1-butynyl, 2-butynyl, and 3-butynyl groups.

The term “cycloalkyl” refers to a hydrocarbon group selected from saturated and partially unsaturated cyclic hydrocarbon groups, comprising monocyclic and polycyclic (e.g., bicyclic and tricyclic) groups. For example, the cycloalkyl group may be of 3-12, or 3-8, or 3-6 carbon atoms. Even further for example, the cycloalkyl group may be a monocyclic group of 3-12, or 3-8, or 3-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4, 4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e. partially unsaturated), but is not fully conjugated, and is not aromatic, as aromatic is defined herein.

The term “aryl” herein refers to a group selected from: 5- and 6-membered carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group is selected from 5- and 6-membered carbocyclic aromatic rings fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “halogen” or “halo” refers to F, Cl, Br or I.

The term “heteroalkyl” refers to alkyl comprising at least one heteroatom.

The term “heteroaryl” refers to a group selected from:
5- to 7-membered aromatic, monocyclic rings comprising 1, 2, 3 or 4 heteroatoms selected from N, O, and S, with the remaining ring atoms being carbon;
8- to 12-membered bicyclic rings comprising 1, 2, 3 or 4 heteroatoms, selected from N, O, and S, with the remaining ring atoms being carbon and wherein at least one ring is aromatic and at least one heteroatom is present in the aromatic ring; and
11- to 14-membered tricyclic rings comprising 1, 2, 3 or 4 heteroatoms, selected from N, O, and S, with the remaining ring atoms being carbon and wherein at least one ring is aromatic and at least one heteroatom is present in an aromatic ring.

For example, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such aslH-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” refers to a ring selected from 4- to 12-membered monocyclic, bicyclic and tricyclic, saturated and partially unsaturated rings comprising at least one carbon atoms in addition to 1, 2, 3 or 4 heteroatoms, selected from oxygen, sulfur, and nitrogen. “Heterocycle” also refers to a 5- to 7-membered heterocyclic ring comprising at least one heteroatom selected from N, O, and S fused with 5-, 6-, and/or 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e. partially unsaturated). The heterocycle may be substituted with oxo. The point of the attachment may be carbon or heteroatom in the heterocyclic ring. A heterocyle is not a heteroaryl as defined herein.

Examples of the heterocycle include, but not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl and 1, 1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicylclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

In embodiments substituents are selected from optionally substituted heteroatom and optionally substituted, optionally hetero-, optionally cyclic C1-C18 hydrocarbyl, particularly wherein the optionally substituted, optionally hetero-, optionally cyclic C1-C18 hydrocarbyl is optionally-substituted, optionally hetero-, optionally cyclic alkyl, alkenyl or alkynyl, or optionally-substituted, optionally hetero-aryl; and/or the optionally substituted heteroatom is halogen, optionally substituted hydroxyl (such as alkoxy, aryloxy), optionally substituted acyl (such as formyl, alkanoyl, carbamoyl, carboxyl, amido), optionally substituted amino (such as amino, alkylamino, dialkylamino, amido, sulfamidyl), optionally substituted thiol (such as mercapto, alkylthiol, aryl thiol), optionally substituted sulfinyl or sulfonyl (such as alkylsulfinyl, arylsulfinyl, alkyl sulfonyl, arylsulfonyl), nitro, or cyano.

In embodiments, substituents are selected from: halogen, —R′, —OR′, ═O, ═NR′, ═N—OR′, —NR′R″, —SR′, —SiR′R″R′″, —OC(O)R′, —C(O)R′, —CO2R′, —CONR′R″, —OC(O)NR′R″, —NR″C(O)R′, —NR′—C(O)NR″R′″, —NR′—SO2NR′″, —NR″CO2R′, —NH—C(NH2)═NH, —NR′C(NH2)═NH, —NH—C(NH2)═NR′, —S(O)R′, —SO2R′, —SO2NR′R″, —NR″SO2R, —CN and —NO2, —N3, —CH(Ph)2, perfluoro(C1-C4)alkoxy and perfluoro(C1-C4)alkyl, in a number ranging from zero to three, with those groups having zero, one or two substituents being particularly preferred. R′, R″ and R′″ each independently refer to hydrogen, unsubstituted (C1-C8)alkyl and heteroalkyl, (C1-C8)alkyl and heteroalkyl substituted with one to three halogens, unsubstituted aryl, aryl substituted with one to three halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl, “alkyl” includes groups such as trihaloalkyl (e.g., —CF3 and —CH2CF3), and when the aryl group is 1,2,3,4-tetrahydronaphthalene, it may be substituted with a substituted or unsubstituted (C3-C7)spirocycloalkyl group. The (C3-C7)spirocycloalkyl group may be substituted in the same manner as defined herein for “cycloalkyl”.

Preferred substituents are selected from: halogen, —R′, —OR′, ═O, —NR′R″, —SR′, —SiR′R″R′″, —OC(O)R′, —C(O)R′, —CO2R′, —CONR′R″, —OC(O)NR′R″, —NR″C(O)R′, —NR″CO2R′, —NR′—SO2NR″R′″, —S(O)R′, —SO2R′, —SO2NR′R″, —NR″SO2R, —CN and —NO2, perfluoro(C1-C4)alkoxy and perfluoro(C1-C4)alkyl, where R′ and R″ are as defined above.

Preferred substituents are disclosed herein and exemplified in the tables, structures, examples, and claims, and may be applied across different compounds of the invention, i.e. substituents of any given compound may be combinatorially used with other compounds.

In particular embodiments applicable substituents are independently substituted or unsubstituted heteroatom, substituted or unsubstituted, 0-3 heteroatom C1-C6 alkyl, substituted or unsubstituted, 0-3 heteroatom C2-C6 alkenyl, substituted or unsubstituted, 0-3 heteroatom C2-C6 alkynyl, or substituted or unsubstituted, 0-3 heteroatom C6-C14 aryl, wherein each heteroatom is independently oxygen, phosphorus, sulfur or nitrogen.

In more particular embodiments, applicable substituents are independently aldehyde, aldimine, alkanoyloxy, alkoxy, alkoxycarbonyl, alkyloxy, alkyl, amine, azo, halogens, carbamoyl, carbonyl, carboxamido, carboxyl, cyanyl, ester, halo, haloformyl, hydroperoxyl, hydroxyl, imine, isocyanide, iscyante, N-tert-butoxycarbonyl, nitrate, nitrile, nitrite, nitro, nitroso, phosphate, phosphono, sulfide, sulfonyl, sulfo, sulfhydryl, thiol, thiocyanyl, trifluoromethyl or trifluromethyl ether (OCF3).

The compounds may contain an asymmetric center and may thus exist as enantiomers. Where the compounds possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds contain olefin double bonds, unless specified otherwise, such double bonds are meant to include both E and Z geometric isomers.

Some of the compounds may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents. Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, aluminum, lithium, and ammonium.

In addition, if a compound is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

“Treating,” “treat,” or “treatment” refers to administering at least one compound and/or at least one stereoisomer thereof, and/or at least one pharmaceutically acceptable salt thereof to a subject in recognized need thereof.

An “effective amount” refers to an amount of at least one compound and/or at least one stereoisomer thereof, and/or at least one pharmaceutically acceptable salt thereof effective to “treat” a disease or disorder in a subject, and that will elicit, to some significant extent, the biological or medical response of a tissue, system, animal or human that is being sought, such as when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The term “at least one substituent” includes, for example, from 1 to 4, such as from 1 to 3, further as 1 or 2, substituents. For example, “at least one substituent Rie” herein includes from 1 to 4, such as from 1 to 3, further as 1 or 2, substituents selected from the list of R′⁶ as described herein.

The subject compounds and stereoisomers thereof, and pharmaceutically acceptable salts thereof may be employed alone or in combination with at least one other therapeutic agent for treatment. In some embodiments, the compounds, stereoisomers thereof, and pharmaceutically acceptable salts thereof can be used in combination with at least one additional therapeutic agent. The compound and/or one pharmaceutically acceptable salt disclosed herein may be administered with the at least one other therapeutic agent in a single dosage form or as a separate dosage form. When administered as a separate dosage form, the at least one other therapeutic agent may be administered prior to, at the same time as, or following administration of the compound and/or one pharmaceutically acceptable salt disclosed herein.

Also provided is a composition comprising a subject compound and stereoisomers thereof, and pharmaceutically acceptable salts thereof, and at least one pharmaceutically acceptable carrier.

The composition comprising a subject compound and stereoisomers thereof, and pharmaceutically acceptable salts thereof can be administered in various known manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art.

The subject compounds and stereoisomers thereof, and pharmaceutically acceptable salts thereof can be administered orally in solid dosage forms, such as capsules, tablets, troches, dragées, granules and powders, or in liquid dosage forms, such as elixirs, syrups, emulsions, dispersions, and suspensions. The subject compounds and stereoisomers thereof, and pharmaceutically acceptable salts thereof disclosed herein can also be administered parenterally, in sterile liquid dosage forms, such as dispersions, suspensions or solutions. Other dosages forms that can also be used to administer the subject compounds and stereoisomers thereof, and pharmaceutically acceptable salts thereof disclosed herein as an ointment, cream, drops, transdermal patch or powder for topical administration, as an ophthalmic solution or suspension formation, i.e., eye drops, for ocular administration, as an aerosol spray or powder composition for inhalation or intranasal administration, or as a cream, ointment, spray or suppository for rectal or vaginal administration.

Gelatin capsules containing the compound and/or the at least one pharmaceutically acceptable salt thereof disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene gycols can be examples of suitable carriers for parenteral solutions. Solutions for parenteral administration may comprise a water soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients.

Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol, a standard reference text in the art.

For administration by inhalation, the subject compounds and stereoisomers thereof, and pharmaceutically acceptable salts thereof may be conveniently delivered in the form of an aerosol spray presentation from pressurized packs or nebulisers. The subject compounds and stereoisomers thereof, and pharmaceutically acceptable salts thereof may also be delivered as powders, which may be formulated and the powder composition may be inhaled with the aid of an insufflation powder inhaler device. One exemplary delivery system for inhalation can be metered dose inhalation (MDI) aerosol, which may be formulated as a suspension or solution of a subject compound and stereoisomers thereof, and pharmaceutically acceptable salts thereof disclosed herein in at least one suitable propellant, selected, for example, from fluorocarbons and hydrocarbons.

For ocular administration, an ophthalmic preparation may be formulated with an appropriate weight percentage of a solution or suspension of the subject compound and stereoisomers thereof, and pharmaceutically acceptable salts thereof in an appropriate ophthalmic vehicle, such that the subject compound and stereoisomers thereof, and at least one pharmaceutically acceptable salts thereof is maintained in contact with the ocular surface for a sufficient time period to allow the compound to penetrate the corneal and internal regions of the eye.

Useful pharmaceutical dosage-forms for administration of the subject compounds and stereoisomers thereof, and pharmaceutically acceptable salts thereof disclosed herein include, but are not limited to, hard and soft gelatin capsules, tablets, parenteral injectables, and oral suspensions.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, a large number of unit capsules can be prepared by filling standard two-piece hard gelatin capsules each with, for example, 100 milligrams of the subject compound and stereoisomers thereof, and pharmaceutically acceptable salt thereof disclosed herein in powder, 150 milligrams of lactose, 50 milligrams of cellulose, and 6 milligrams magnesium stearate.

In some embodiments, a mixture of the compound, stereoisomers thereof, and pharmaceutically acceptable salts thereof a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, a large number of tablets can be prepared by conventional procedures so that the dosage unit comprises, for example, 100 milligrams of the compound, stereoisomers thereof, and pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, and pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, stereoisomers thereof, and pharmaceutically acceptable salts thereof are administered stepwise or in conjunction with at least one other therapeutic agent. When drugs are administered in physical combination, the dosage form and administration route should be selected depending on the compatibility of the combined drugs. Thus the term coadministration is understood to include the administration of at least two agents concomitantly or sequentially, or alternatively as a fixed dose combination of the at least two active components.

The compounds, stereoisomers thereof, and pharmaceutically acceptable salt thereof disclosed herein can be administered as the sole active ingredient or in combination with at least one second active ingredient.

The subject compounds are incorporated into pharmaceutical compositions or formulations. The compositions will contain pharmaceutically acceptable diluents and/or carriers, i. e. diluents or carriers that are physiologically compatible and substantially free from pathogenic impurities. Suitable excipients or carriers and methods for preparing administrable compositions are known or apparent to those skilled in the art and are described in more detail in such publications as Remington's Pharmaceutical Science, Mack Publishing Co, NJ (1991). The compositions may also be in the form of controlled release or sustained release compositions as known in the art. For many applications the subject compounds are administered for morning/daytime dosing, with off period at night.

The subject compounds may be used per se, or in the form of their pharmaceutically acceptable salts, such as hydrochlorides, hydrobromides, acetates, sulfates, citrates, carbonates, trifluoroacetates and the like. When compounds contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When compounds contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al, “Pharmaceutical Salts”, Journal of Pharmaceutical Science, 1977, 66, 1-19).

The neutral forms of the compounds may be regenerated by contacting the salt with a base or acid, and isolating the parent compound in the conventional manner. The parent form of the compound differs from the various salt forms in certain physical properties, such as solubility in polar solvents, but otherwise the salts are equivalent to the parent form of the compound for the purposes of this invention.

In addition to salt forms, this invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity.

Certain compounds of the invention can exist in unsolvated forms as well as solvated forms, including hydrated forms. In general, the solvated forms are equivalent to unsolvated forms and are intended to be encompassed within the scope of the present invention. Certain compounds of the invention may exist in multiple crystalline or amorphous forms. In general, all physical forms are equivalent for the uses contemplated by the present invention and are intended to be within the scope of the invention.

Some of the subject compounds possess asymmetric carbon atoms (optical centers) or double bonds; the racemates, diastereomers, geometric isomers and individual isomers are all intended to be encompassed within the scope of the present invention.

The compounds of the invention may also contain unnatural proportions of atomic isotopes at one or more of the atoms that constitute such compounds, such as deuterium, e.g. —CD₃, CD₂H or CDH₂ in place of methyl. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

The compounds are generally administered in a “therapeutically effective amount”, i.e. the amount of the subject compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The contacting is generally effected by administering to the subject an effective amount of one or more compounds having the general formula I (supra), including the various embodiments described above. Generally administration is adjusted to achieve a therapeutic dosage of about 0.1 to 50, preferably 0.5 to 10, more preferably 1 to 10 mg/kg, though optimal dosages are compound specific, and generally empirically determined for each compound.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the mimetic is usually a minor component (from about 0.1 to about 50% by weight or preferably from about 1 to about 40% by weight) with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

The subject compositions may also be coformulated and/or coadministered with a different compound to treat applicable indications, to inhibit ferroptosis activity, or modulate or inhibit a disease associated with ferroptosis dysregulation, such as neuropathy, ischemia reperfusion injury, acute kidney failure and cancer. In embodiments applicable indications include cancer, neuropathy and neurodegenerative disease of the central or peripheral nervous system, muscular dystrophy, ischemia and ischemia reperfusion injury, kidney disease and failure, degenerative arthritis, retinal necrosis, heart disease, liver, gastrointestinal or pancreatic disease, avascular necrosis, diabetes, cancer-chemo/radiation therapy-induced cell-death and intoxication.

TABLE
Active Compounds: Structures

| | |
|---|---|
| 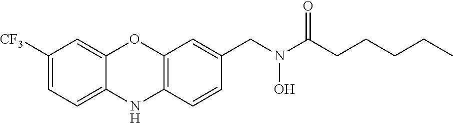 [SMILES: CCCCCC(=O)N(O)Cc1ccc2c(c1)Oc1cc(C(F)(F)F)ccc1N2] | 1 |
| 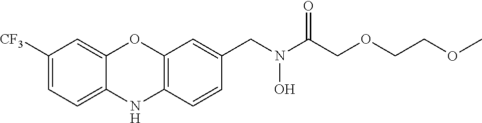 [SMILES: COCCOCC(=O)N(O)Cc1ccc2c(c1)Oc1cc(C(F)(F)F)ccc1N2] | 2 |
| 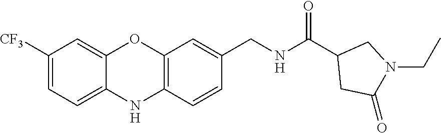 [SMILES: CCN1CC(C(=O)NCc2ccc3c(c2)Oc2cc(C(F)(F)F)ccc2N3)CC1=O] | 3 |
| 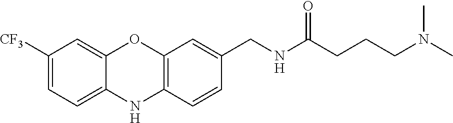 [SMILES: CN(C)CCCC(=O)NCc1ccc2c(c1)Oc1cc(C(F)(F)F)ccc1N2] | 4 |
| 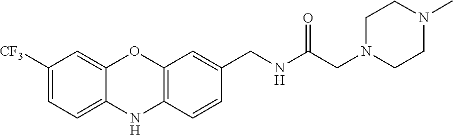 [SMILES: CN1CCN(CC(=O)NCc2ccc3c(c2)Oc2cc(C(F)(F)F)ccc2N3)CC1] | 5 |
| 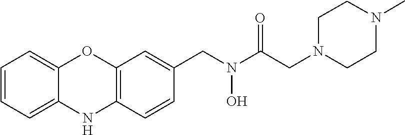 [SMILES: CN1CCN(CC(=O)N(O)Cc2ccc3c(c2)Oc2ccccc2N3)CC1] | 6 |
| 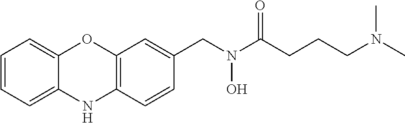 [SMILES: CN(C)CCCC(=O)N(O)Cc1ccc2c(c1)Oc1ccccc1N2] | 7 |
| 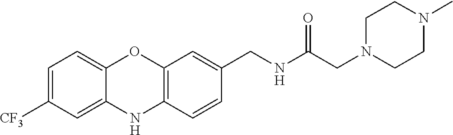 [SMILES: CN1CCN(CC(=O)NCc2ccc3c(c2)Oc2ccc(C(F)(F)F)cc2N3)CC1] | 8 |
| 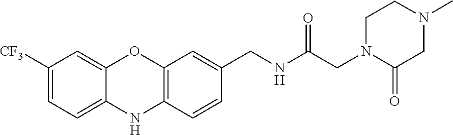 [SMILES: CN1CCN(CC(=O)NCc2ccc3c(c2)Oc2cc(C(F)(F)F)ccc2N3)C(=O)C1] | 9 |
| 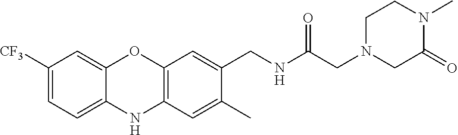 [SMILES: Cc1cc2c(cc1CNC(=O)CN1CCN(C)C(=O)C1)Oc1cc(C(F)(F)F)ccc1N2] | 10 |
| 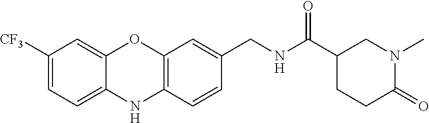 [SMILES: CN1CC(C(=O)NCc2ccc3c(c2)Oc2cc(C(F)(F)F)ccc2N3)CCC1=O] | 11 |
| 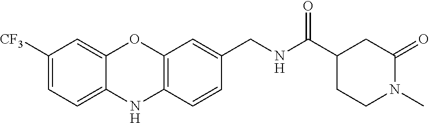 [SMILES: CN1CCC(C(=O)NCc2ccc3c(c2)Oc2cc(C(F)(F)F)ccc2N3)CC1=O] | 12 |
| 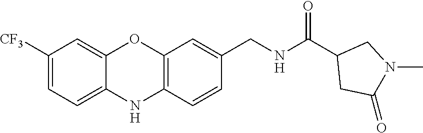 [SMILES: CN1CC(C(=O)NCc2ccc3c(c2)Oc2cc(C(F)(F)F)ccc2N3)CC1=O] | 13 |
| 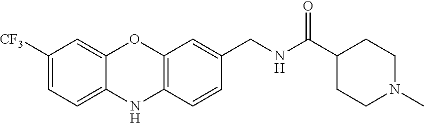 [SMILES: CN1CCC(C(=O)NCc2ccc3c(c2)Oc2cc(C(F)(F)F)ccc2N3)CC1] | 14 |
| 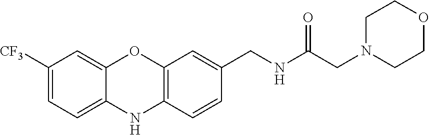 [SMILES: O=C(CN1CCOCC1)NCc1ccc2c(c1)Oc1cc(C(F)(F)F)ccc1N2] | 15 |
| 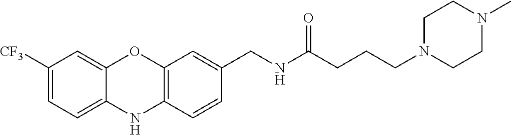 [SMILES: CN1CCN(CCCC(=O)NCc2ccc3c(c2)Oc2cc(C(F)(F)F)ccc2N3)CC1] | 16 |
| 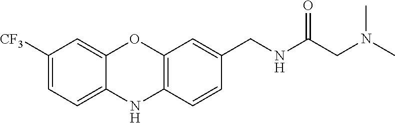 [SMILES: CN(C)CC(=O)NCc1ccc2c(c1)Oc1cc(C(F)(F)F)ccc1N2] | 17 |
| 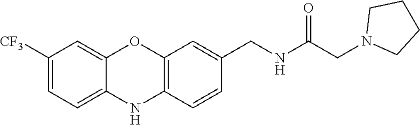 [SMILES: O=C(CN1CCCC1)NCc1ccc2c(c1)Oc1cc(C(F)(F)F)ccc1N2] | 18 |
| 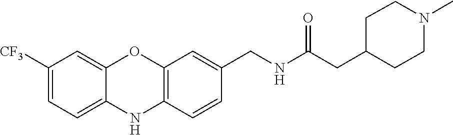 [SMILES: CN1CCC(CC(=O)NCc2ccc3c(c2)Oc2cc(C(F)(F)F)ccc2N3)CC1] | 19 |
| 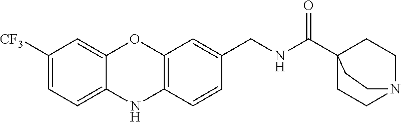 [SMILES: O=C(NCc1ccc2c(c1)Oc1cc(C(F)(F)F)ccc1N2)C12CCN(CC1)CC2] | 20 |
| 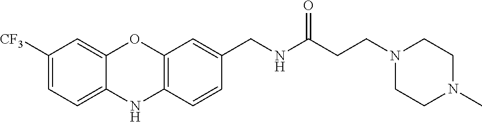 [SMILES: CN1CCN(CCC(=O)NCc2ccc3c(c2)Oc2cc(C(F)(F)F)ccc2N3)CC1] | 21 |
| 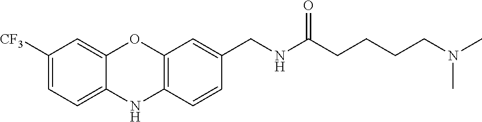 [SMILES: CN(C)CCCCC(=O)NCc1ccc2c(c1)Oc1cc(C(F)(F)F)ccc1N2] | 22 |
| 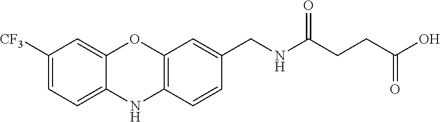 [SMILES: O=C(O)CCC(=O)NCc1ccc2c(c1)Oc1cc(C(F)(F)F)ccc1N2] | 23 |
| 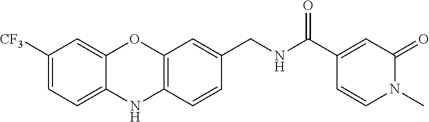 [SMILES: Cn1ccc(C(=O)NCc2ccc3c(c2)Oc2cc(C(F)(F)F)ccc2N3)cc1=O] | 24 |
| 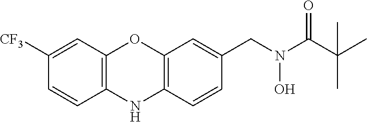 [SMILES: CC(C)(C)C(=O)N(O)Cc1ccc2c(c1)Oc1cc(C(F)(F)F)ccc1N2] | 25 |
| 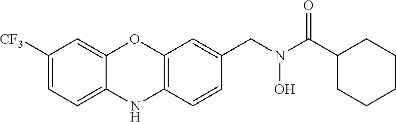 [SMILES: O=C(C1CCCCC1)N(O)Cc1ccc2c(c1)Oc1cc(C(F)(F)F)ccc1N2] | 26 |
| 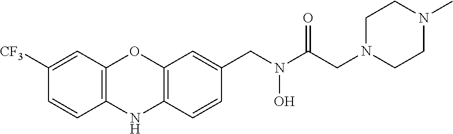 [SMILES: CN1CCN(CC(=O)N(O)Cc2ccc3c(c2)Oc2cc(C(F)(F)F)ccc2N3)CC1] | 27 |
| 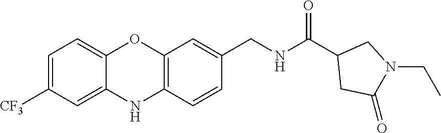 [SMILES: CCN1CC(C(=O)NCc2ccc3c(c2)Oc2ccc(C(F)(F)F)cc2N3)CC1=O] | 28 |
| 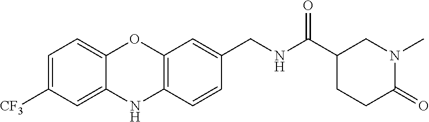 [SMILES: CN1CC(C(=O)NCc2ccc3c(c2)Oc2ccc(C(F)(F)F)cc2N3)CCC1=O] | 29 |
| 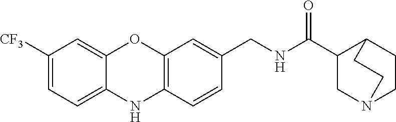 [SMILES: O=C(NCc1ccc2c(c1)Oc1cc(C(F)(F)F)ccc1N2)C1CN2CCC1CC2] | 30 |
| 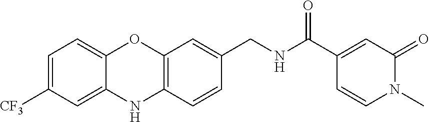 [SMILES: Cn1ccc(C(=O)NCc2ccc3c(c2)Oc2ccc(C(F)(F)F)cc2N3)cc1=O] | 31 |
| 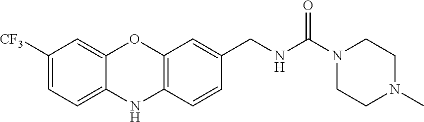 [SMILES: CN1CCN(C(=O)NCc2ccc3c(c2)Oc2cc(C(F)(F)F)ccc2N3)CC1] | 32 |
| 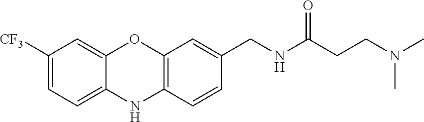 [SMILES: CN(C)CCC(=O)NCc1ccc2c(c1)Oc1cc(C(F)(F)F)ccc1N2] | 33 |
| 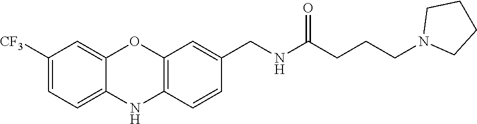 [SMILES: O=C(CCCN1CCCC1)NCc1ccc2c(c1)Oc1cc(C(F)(F)F)ccc1N2] | 34 |
| 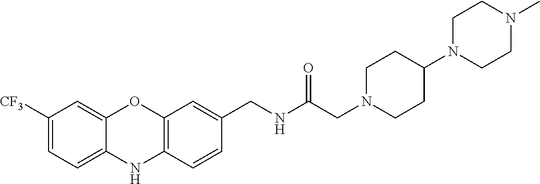 [SMILES: CN1CCN(C2CCN(CC(=O)NCc3ccc4c(c3)Oc3cc(C(F)(F)F)ccc3N4)CC2)CC1] | 35 |
| 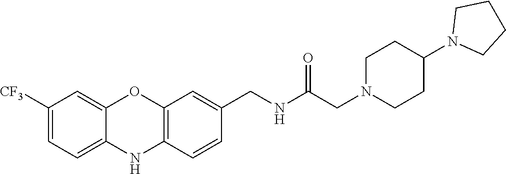 [SMILES: O=C(CN1CCC(N2CCCC2)CC1)NCc1ccc2c(c1)Oc1cc(C(F)(F)F)ccc1N2] | 36 |
| 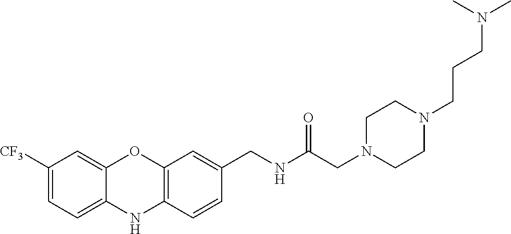 [SMILES: CN(C)CCCN1CCN(CC(=O)NCc2ccc3c(c2)Oc2cc(C(F)(F)F)ccc2N3)CC1] | 37 |
| 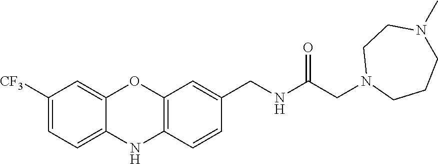 [SMILES: CN1CCCN(CC(=O)NCc2ccc3c(c2)Oc2cc(C(F)(F)F)ccc2N3)CC1] | 38 |
| 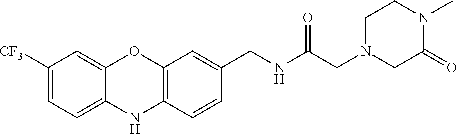 [SMILES: CN1CCN(CC(=O)NCc2ccc3c(c2)Oc2cc(C(F)(F)F)ccc2N3)CC1=O] | 39 |
| 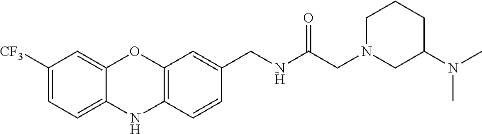 [SMILES: CN(C)C1CCCN(CC(=O)NCc2ccc3c(c2)Oc2cc(C(F)(F)F)ccc2N3)C1] | 40 |
| 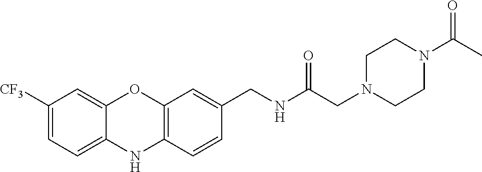 [SMILES: CC(=O)N1CCN(CC(=O)NCc2ccc3c(c2)Oc2cc(C(F)(F)F)ccc2N3)CC1] | 41 |
| 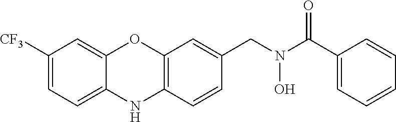 [SMILES: O=C(c1ccccc1)N(O)Cc1ccc2c(c1)Oc1cc(C(F)(F)F)ccc1N2] | 42 |
| 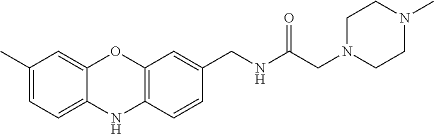 | 43 |
| 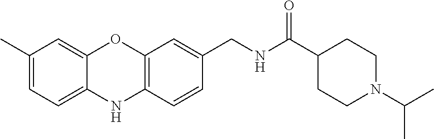 [SMILES: Cc1ccc2c(c1)Oc1cc(CNC(=O)C3CCN(C(C)C)CC3)ccc1N2] | 44 |
| 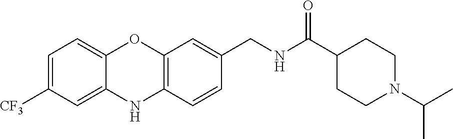 [SMILES: CC(C)N1CCC(C(=O)NCc2ccc3c(c2)Oc2ccc(C(F)(F)F)cc2N3)CC1] | 45 |
| 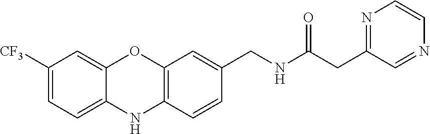 [SMILES: O=C(Cc1cnccn1)NCc1ccc2c(c1)Oc1cc(C(F)(F)F)ccc1N2] | 46 |
| 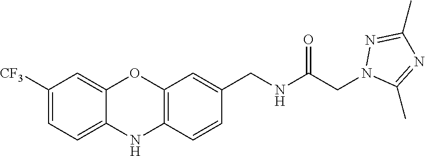 [SMILES: Cc1nc(C)n(CC(=O)NCc2ccc3c(c2)Oc2cc(C(F)(F)F)ccc2N3)n1] | 47 |
| 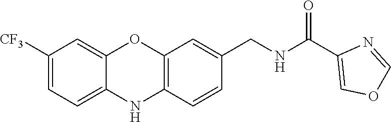 [SMILES: O=C(NCc1ccc2c(c1)Oc1cc(C(F)(F)F)ccc1N2)c1cocn1] | 48 |
| 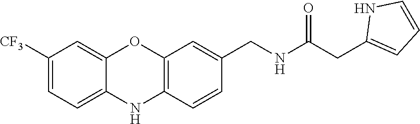 [SMILES: O=C(Cc1ccc[nH]1)NCc1ccc2c(c1)Oc1cc(C(F)(F)F)ccc1N2] | 49 |
| 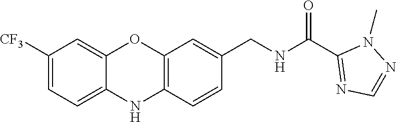 [SMILES: Cn1ncnc1C(=O)NCc1ccc2c(c1)Oc1cc(C(F)(F)F)ccc1N2] | 50 |
| 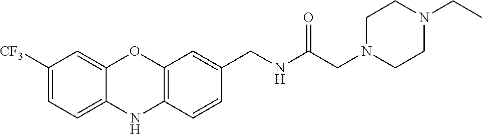 [SMILES: CCN1CCN(CC(=O)NCc2ccc3c(c2)Oc2cc(C(F)(F)F)ccc2N3)CC1] | 51 |
| 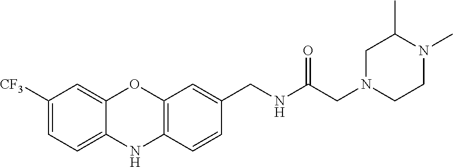 [SMILES: CC1CN(CC(=O)NCc2ccc3c(c2)Oc2cc(C(F)(F)F)ccc2N3)CCN1C] | 52 |
| 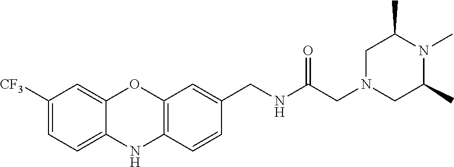 [SMILES: C[C@@H]1CN(CC(=O)NCc2ccc3c(c2)Oc2cc(C(F)(F)F)ccc2N3)C[C@H](C)N1C] | 53 |
| 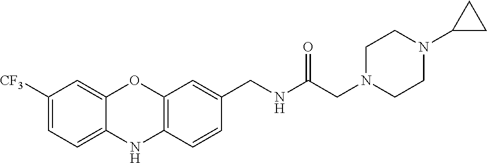 [SMILES: O=C(CN1CCN(C2CC2)CC1)NCc1ccc2c(c1)Oc1cc(C(F)(F)F)ccc1N2] | 54 |
| 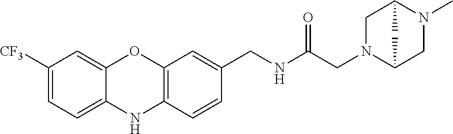 [SMILES: CN1C[C@@H]2C[C@H]1CN2CC(=O)NCc1ccc2c(c1)Oc1cc(C(F)(F)F)ccc1N2] | 55 |
| 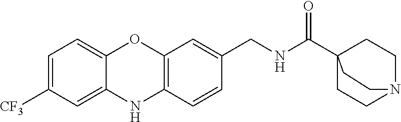 [SMILES: O=C(NCc1ccc2c(c1)Oc1ccc(C(F)(F)F)cc1N2)C12CCN(CC1)CC2] | 56 |
| 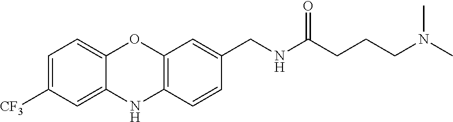 [SMILES: CN(C)CCCC(=O)NCc1ccc2c(c1)Oc1ccc(C(F)(F)F)cc1N2] | 57 |
| 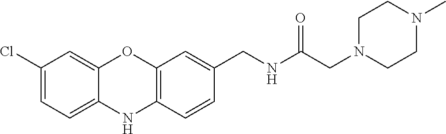 [SMILES: CN1CCN(CC(=O)NCc2ccc3c(c2)Oc2cc(Cl)ccc2N3)CC1] | 58 |
| 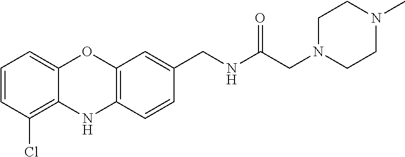 [SMILES: CN1CCN(CC(=O)NCc2ccc3c(c2)Oc2cccc(Cl)c2N3)CC1] | 59 |
| 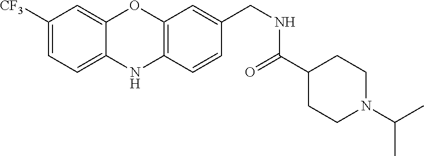 [SMILES: CC(C)N1CCC(C(=O)NCc2ccc3c(c2)Oc2cc(C(F)(F)F)ccc2N3)CC1] | 60 |
| 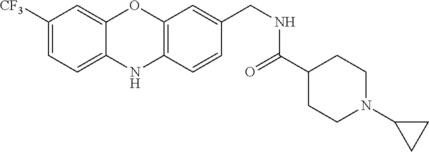 [SMILES: O=C(NCc1ccc2c(c1)Oc1cc(C(F)(F)F)ccc1N2)C1CCN(C2CC2)CC1] | 61 |
| 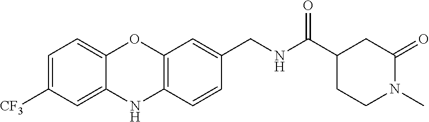 [SMILES: CN1CCC(C(=O)NCc2ccc3c(c2)Oc2ccc(C(F)(F)F)cc2N3)CC1=O] | 62 |
| 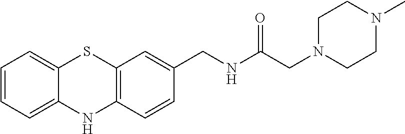 [SMILES: CN1CCN(CC(=O)NCc2ccc3c(c2)Sc2ccccc2N3)CC1] | 63 |

Active compounds are demonstrated to inhibit ferroptosis:

TABLE
Bioactivity (RSL3-induced HT-1080
cells ferroptosis assay (10% FBS):

| # | | # | | # | | # | |
|---|---|---|---|---|---|---|---|
| 1 | 1-100 nM | 2 | 1-100 nM | 3 | 1-100 nM | 4 | 1-100 nM |
| 5 | 1-100 nM | 6 | 1-100 nM | 7 | 1-100 nM | 8 | 1-100 nM |
| 9 | 1-100 nM | 10 | 1-100 nM | 11 | 1-100 nM | 12 | 1-100 nM |
| 13 | 1-100 nM | 14 | 1-100 nM | 15 | 1-100 nM | 16 | 1-100 nM |
| 17 | 1-100 nM | 18 | 1-100 nM | 19 | 1-100 nM | 20 | 1-100 nM |
| 21 | 1-100 nM | 22 | 1-100 nM | 23 | 1-100 nM | 24 | 1-100 nM |
| 25 | 1-100 nM | 26 | 1-100 nM | 27 | 1-100 nM | 28 | 1-100 nM |
| 29 | 1-100 nM | 30 | 1-100 nM | 31 | 1-100 nM | 32 | 1-100 nM |
| 33 | 1-100 nM | 34 | 1-100 nM | 35 | 1-100 nM | 36 | 1-100 nM |
| 37 | 1-100 nM | 38 | 1-100 nM | 39 | 1-100 nM | 40 | 1-100 nM |
| 41 | 1-100 nM | 42 | 1-100 nM | 43 | 1-100 nM | 44 | 1-100 nM |
| 45 | 1-100 nM | 46 | 1-100 nM | 47 | 1-100 nM | 48 | 1-100 nM |
| 49 | 1-100 nM | 50 | 1-100 nM | 51 | 1-100 nM | 52 | 1-100 nM |
| 53 | 1-100 nM | 54 | 1-100 nM | 55 | 1-100 nM | 56 | 1-100 nM |
| 57 | 1-100 nM | 58 | 1-100 nM | 59 | 1-100 nM | 60 | 1-100 nM |
| 61 | 1-100 nM | 62 | 1-100 nM | 63 | 1-100 nM | | |

### Active Compounds: Representative Synthesis

### N-hydroxy-N-((7-(trifluoromethyl)-10H-phenoxazin-3-yl)methyl)hexanamide (1)

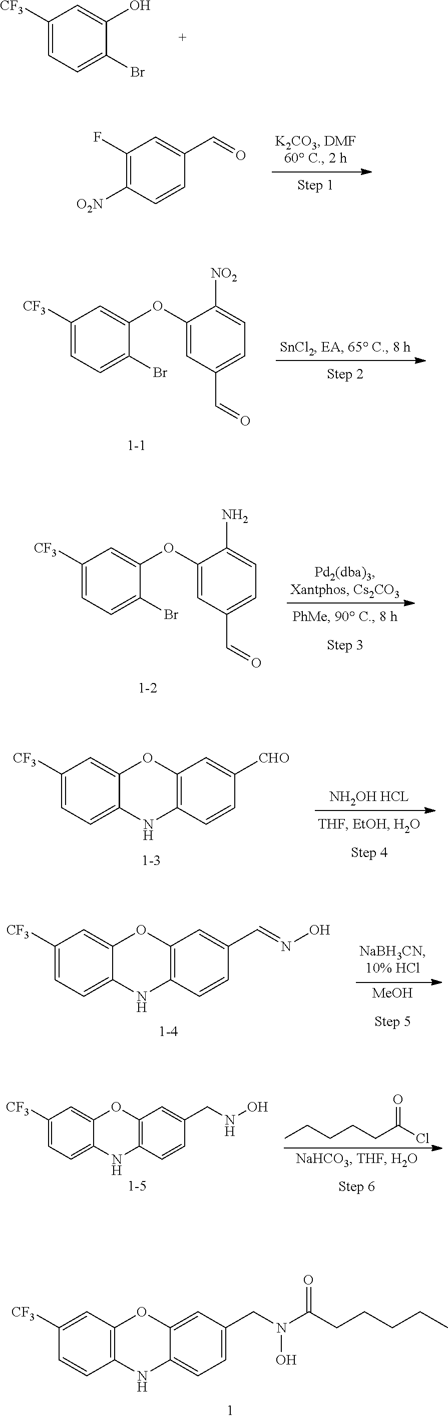

Step 1. A mixture solution of 2-bromo-5-(trifluoromethyl)phenol (20 g, 82.99 mmol), 3-fluoro-4-nitrobenzaldehyde (15.44 g, 91.28 mmol), K₂CO₃ (34.41 g, 248.96 mmo) in DMF (500 mL) was stirred at 60° C. for 2 h. The mixture was concentrated under reduced pressure, the residue was purified by flash, eluting with PE: EA=10:1 to 1:1 to give the desired product as yellow solid (12 g, 37.5%).

Step 2. To a solution of 3-(2-bromo-5-(trifluoromethyl)phenoxy)-4-nitrobenzaldehyde (12 g, 30.76 mmol) in EA (300 mL) was added SnCl₂ (34.99 g, 184.56 mmol, then the mixture was stirred at 65° C. for 8 h. to the mixture was added NaHCO₃ (sat.) (300 mL), then the mixture was filtered, the filter cake was washed by EA (100 mL×3). The combined organic layers were dried over Na₂SO₄, concentrated and purified by flash, eluting with PE: EA=10:1 to 1:1 to give the product as yellow solid (9.4 g, 84.8%). Mass(m/z): 359.6 [M+H]⁺.

Step 3. A mixture of 4-amino-3-(2-bromo-5-(trifluoromethyl)phenoxy)benzaldehyde (8 g, 22.21 mmol), Pd₂(dba)₃ (2.03 g, 2.22 mmol), Xantphos (2.56 g, 4.44 mmol), Cs₂CO₃ (21.82 g, 66.6 mmo) and Tol (1200 mL) was stirred at 90° C. under N2 atmosphere for 8 h. The mixture was concentrated and purified by flash, eluting with PE: EA=1:0 to 1:1 to give the desired product as white solid (3.2 g, 51.6%). ¹H NMR (300 MHz, DMSO-d₆) δ 9.61 (s, 1H), 9.42 (s, 1H), 7.35 (dd, J=8.0, 1.7 Hz, 1H), 7.10 (d, J=8.2 Hz, 1H), 7.00 (d, J=1.6 Hz, 1H), 6.92 (s, 1H), 6.59 (dd, J=8.0, 5.2 Hz, 2H). Mass(m/z): 279.8 [M+H]⁺.

Step 4. To a solution of 7-(trifluoromethyl)-10H-phenoxazine-3-carbaldehyde (600 mg, 2.150 mmol) in EtOH (15 mL), THF (6 mL) and water (3 mL) was added Hydroxylamine hydrochloride (224 mg, 3.226 mmol). Then the reaction was stirred overnight at rt. The reaction mixture was concentrated under vacuum. The crude was used directly at next step. (100%).

Mass(m/z): 295.1 [M+H]⁺.

Step 5. To a solution of E)-7-(trifluoromethyl)-10H-phenoxazine-3-carbaldehyde oxime (700 mg, 2.381 mmol) in MeOH (15 mL) was added NaBH₄ (225 mg, 3.572 mmol). Then 10% HCl (5 mL) was added dropwise at 0° C. The solution was stirred for 2 hours at rt. The PH of the solution was adjusted to 8-9 with sodium carbonate solution. Then the mixture was extracted by DCM (15 mL×3). The combined organic layers were washed with water (20 mL×3), dried over Na₂SO₄ and concentrated under vacuum. The residue was purified by silica gel chromatography (MeOH:DCM=1:10) to afford the target product as a yellow solid. (543 mg, 77.0%). Mass(m/z): 264.1 [M−32]⁺.

Step 6. To a solution of N-((7-(trifluoromethyl)-10H-phenoxazin-3-yl)methyl)hydroxylamine (30 mg, 0.101 mmol) and hexanoyl chloride (16 mg, 0.122 mmol) in THF/H₂O (2 mL/2 mL) was added NaHCO₃ (25 mg, 0.303 mmol), then the mixture was stirred at room temperature for 2 h. The mixture was extracted by EA (25 mL×3). The combined organic layers were washed with brine (15 mL×3), dried over Na₂SO₄ and concentrated to give the crude product, which was purified by TLC(MeOH/DCM=15:1) to give the desired product as white solid (2.1 mg, 5.3%). ¹H NMR (400 MHz, Methanol-d₄) δ 6.96 (d, J=7.6 Hz, 1H), 6.75 (d, J=1.8 Hz, 1H), 6.69 (d, J=7.2 Hz, 1H), 6.56 (d, J=1.8 Hz, 1H), 6.47 (d, J=8.0 Hz, 1H), 6.40 (d, J=7.8 Hz, 1H), 4.59 (s, 2H), 2.26 (t, J=7.6 Hz, 2H), 1.65-1.55 (m, 4H), 1.28 (s, 2H), 0.91 (s, 3H). Mass(m/z): 392.9 [M−H]⁺.

### N-hydroxy-2-(2-methoxyethoxy)-N-((7-(trifluoromethyl)-10H-phenoxazin-3-yl)methyl)acetamide (2)

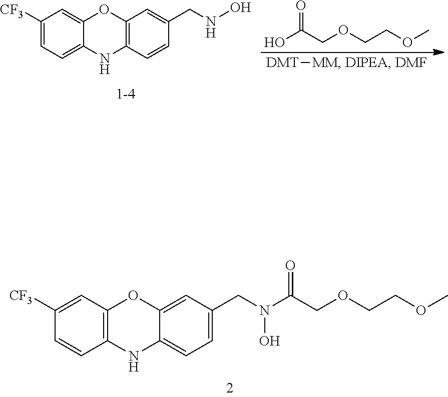

Step 1. To a solution of N-((7-(trifluoromethyl)-10H-phenoxazin-3-yl)methyl)hydroxylamine (30 mg, 0.101 mmol) and 2-(2-methoxyethoxy)acetic acid (18 mg, 0.132 mmol) in DMF (3 mL) was added DMT-MM (39 mg, 0.132 mmol) and DIPEA (17 mg, 0.132 mmol), then the mixture was stirred at room temperature for 2 h. The mixture was extracted by EA (25 mL×3). The combined organic layers were washed with brine (15 mL×3), dried over Na₂SO₄ and concentrated to give the crude product, which was purified by TLC (MeOH/DCM=10:1) to give the desired product as white solid (6.2 mg, 14.8%). ¹H NMR (400 MHz, Methanol-d₄) δ 6.97 (d, J=7.6 Hz, 1H), 6.75 (d, J=2.0 Hz, 1H), 6.72-6.68 (m, 1H), 6.58 (d, J=1.8 Hz, 1H), 6.47 (d, J=8.2 Hz, 1H), 6.40 (d, J=7.8 Hz, 1H), 4.64-4.51 (m, 4H), 4.36 (s, 2H), 3.71-3.68 (m, 2H), 3.60-3.57 (m, 2H), 3.37 (s, 3H). Mass(m/z): 413.2 [M+H]⁺.

### 1-ethyl-5-oxo-N-((7-(trifluoromethyl)-10H-phenoxazin-3-yl)methyl)pyrrolidine-3-carboxamide (3)

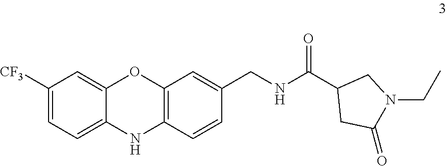 [SMILES: CCN1CC(C(=O)NCc2ccc3c(c2)Oc2cc(C(F)(F)F)ccc2N3)CC1=O]

The title compound 3 (28.2 mg) was prepared in a total yield of 51.7% as a yellow solid from (7-(trifluoromethyl)-10H-phenoxazin-3-yl)methanamine (44 mg, 0.15 mmol), 1-ethyl-5-oxopyrrolidine-3-carboxylic acid (24 mg, 0.15 mmol), DIEA (58 mg, 0.45 mmol) and HATU (57 mg, 0.15 mmol) according to the procedure for 1. ¹H NMR (400 MHz, Methanol-d₄) δ 6.96 (d, J=6.9 Hz, 1H), 6.74 (s, 1H), 6.66 (d, J=7.9 Hz, 1H), 6.54 (s, 1H), 6.45 (d, J=8.2 Hz, 1H), 6.38 (d, J=7.9 Hz, 1H), 4.16 (s, 2H), 3.68-3.52 (m, 2H), 3.39-3.31 (m, 2H), 3.24-3.15 (m, 1H), 2.60 (d, J=8.4 Hz, 2H), 1.12 (t, J=7.2 Hz, 3H). Mass(m/z): 420.2[M+H]⁺.

### 4-(dimethylamino)-N-((7-(trifluoromethyl)-10H-phenoxazin-3-yl)methyl)butanamide (4)

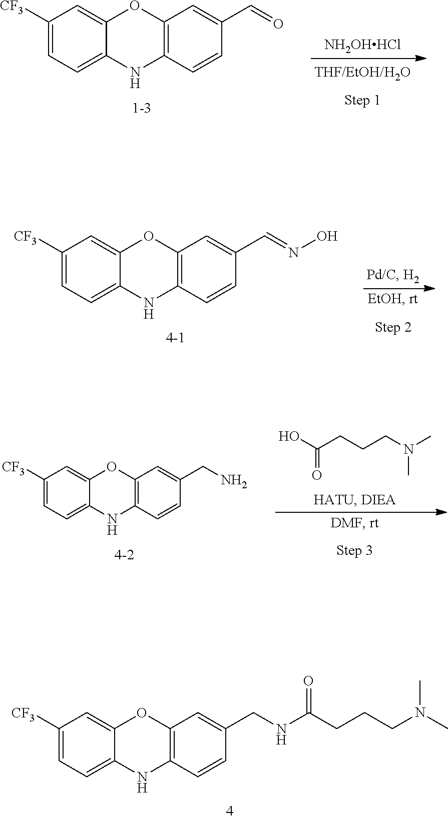

Step 1. To a solution of 7-(trifluoromethyl)-10H-phenoxazine-3-carbaldehyde (1.4 g, 5 mmol) in EtOH (40 mL) was added Hydroxylamine hydrochloride (690 mg). Then the reaction was stirred overnight at rt. The reaction mixture was concentrated under vacuum. The crude was used directly at next step. (100%). Mass(m/z): 295.1 [M+H]⁺.

Step 2. To a solution of (E)-7-(trifluoromethyl)-10H-phenoxazine-3-carbaldehyde oxime (1.0 g, 3.4 mmol) in EtOH (20 mL) was added 10% Pd/C (36 mg, 0.034 ml) and AcOH (0.5 mL). Then the reaction was stirred overnight at rt under an atmosphere of Hydrogen. Pd/C was filtrated out. The PH of the filtration was adjusted to 8-9 with sodium carbonate solution. Then the mixture was extracted by DCM (20 mL×3). The combined organic layers were washed with brine (20 mL×3), dried over Na₂SO₄ and concentrated to give the desired product as yellow solid. (450 mg, 47.4%). 264.1 [M−NH2]⁺.

Step 3. To a solution of (7-(trifluoromethyl)-10H-phenoxazin-3-yl)methanamine (28 mg, 0.1 mmol) and 4-(dimethylamino)butanoic acid. hydrochloride salt (16.7 mg, 0.1 mmol) in DMF (2 ml) was added DIEA (38.7 mg, 0.3 mmol). Followed by the addition of HATU (41.8 mg, 0.11 mmol) then the reaction mixture was stirred for 2 hours at rt. 10 mL of water was added. Then the mixture was extracted by DCM (10 mL×3). The combined organic layers were washed with water (10 mL×3), dried over Na₂SO₄ and concentrated under vacuum. The residue was purified by prep-TLC (MeOH/DCM=1/10) to give the desired product as white solid (10.0 mg, 25.4%). ¹H NMR (400 MHz, Methanol-d₄) δ 6.99-6.94 (m, 1H), 6.76-6.71 (m, 1H), 6.67 (dd, J=8.0, 2.0 Hz, 1H), 6.56 (d, J=2.0 Hz, 1H), 6.50-6.45 (m, 1H), 6.39 (d, J=8.0 Hz, 1H), 4.15 (s, 2H), 3.13-3.04 (m, 2H), 2.84 (s, 6H), 2.40 (t, J=7.0 Hz, 2H), 2.02-1.94 (m, 2H). Mass(m/z): 394.2 [M+H]⁺.

### 2-(4-methylpiperazin-1-yl)-N-((7-(trifluoromethyl)-10H-phenoxazin-3-yl)methyl)acetamide (5)

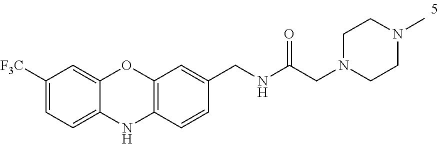 [SMILES: CN1CCN(CC(=O)NCc2ccc3c(c2)Oc2cc(C(F)(F)F)ccc2N3)CC1]

The title compound 5 (15 mg) was prepared in a total yield of 35.7% as a white solid from (7-(trifluoromethyl)-10H-phenoxazin-3-yl)methanamine (28 mg, 0.1 mmol), 2-(4-methylpiperazin-1-yl)acetic acid (15.8 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 6.99-6.95 (m, 1H), 6.74 (dd, J=2.0, 0.6 Hz, 1H), 6.69 (dd, J=8.0, 2.0 Hz, 1H), 6.57 (d, J=2.0 Hz, 1H), 6.47 (dd, J=8.2, 0.8 Hz, 1H), 6.39 (d, J=8.0 Hz, 1H), 4.21 (s, 2H), 3.29-3.09 (m, 6H), 2.97-2.60 (m, 7H). Mass(m/z): 421.2 [M+H]⁺.

### N-((10H-phenoxazin-3-yl)methyl)-N-hydroxy-2-(4-methylpiperazin-1-yl)acetamide (6)

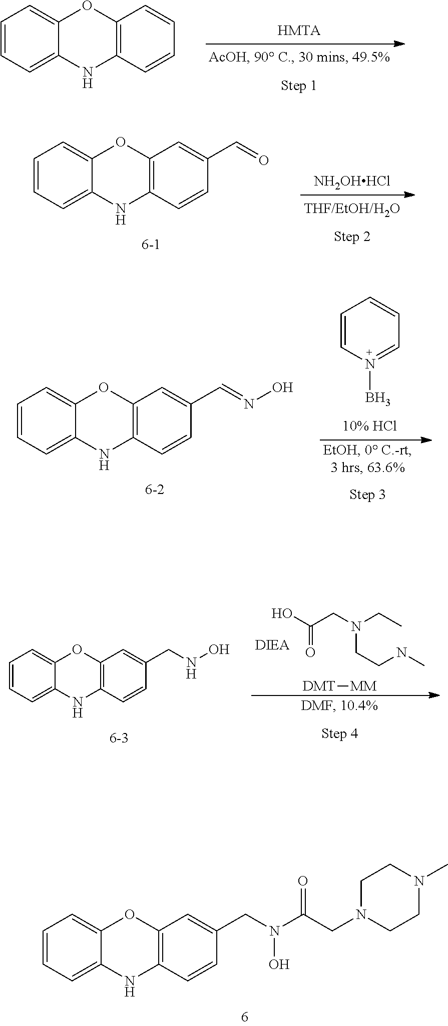

Step 1. To a solution of 10H-phenoxazine (1.83 g, 10 mmol) and AcOH (20 mL) was added Hexamethylenetetramine (1.4 g, 10 mmol). Then the mixture was stirred for mins at 90° C. After cooling to rt. 30 ml of water was added. Then the mixture was extracted by DCM (30 mL×3). The combined organic layers were washed with water (30 mL×3), dried over Na₂SO₄ and concentrated under vacuum to afford desired product as a crude as a yellow solid. (1.05 g, 49.5%). Mass(m/z): 212.2 [M+H]⁺.

Step 2. To a solution of 10H-phenoxazine-3-carbaldehyde (1.05 g, 5 mmol) in a solution of THF/EtOH/H₂O (2/5/1, 32 mL) was added Hydroxylamine hydrochloride (690 mg, 10 mmol). Then the reaction was stirred for 2 hours at rt. The reaction mixture was concentrated under vacuum to added the crude product as a yellow solid. The crude was used directly at next step. (100%). Mass(m/z): 227.1 [M+H]⁺.

Step 3. To a solution of (E)-10H-phenoxazine-3-carbaldehyde oxime (110 mg, 0.5 mmol) in EtOH (15 mL) was added Borane-pyridine (93 mg, 1.0 mmol). Then 10% HCl (5 mL) was added dropwise at 0° C. The solution was stirred for 3 hours at rt. The PH of the solution was adjusted to 8-9 with sodium carbonate solution. Then the mixture was extracted by DCM (15 mL×3). The combined organic layers were washed with water (20 mL×3), dried over Na₂SO₄ and concentrated under vacuum. The residue was purified by prep-TLC MeOH/DCM (0-1/20) to afford the target product as a yellow solid. (70 mg, 63.6%). Mass(m/z): 229.1 [M+H]⁺.

Step 4. To a solution of N-((10H-phenoxazin-3-yl)methyl)hydroxylamine (30 mg, 0.13 mmol), 2-(4-methylpiperazin-1-yl)acetic acid (20.7 mg, 0.13 mmol) and DIEA (50.3 mg, 0.39 mmol) in DMF (1 ml) was added DMT-MM (39.5 mg, 0.14 mmol) then the reaction mixture was stirred for 3 hours at rt. 10 ml of water was added. Then the mixture was extracted by DCM (10 mL×3). The combined organic layers were washed with water (10 mL×3), dried over Na₂SO₄ and concentrated under vacuum. The residue was purified by prep-TLC (MeOH/DCM=1/20) to give the desired product as yellow solid (5.0 mg, 10.4%). 1H NMR (400 MHz, Methanol-d₄) δ 6.75-6.65 (m, 2H), 6.60-6.48 (m, 3H), 6.44-6.33 (m, 2H), 4.53 (s, 2H), 3.63 (s, 2H), 3.34-3.28 (m, 4H), 2.87 (s, 7H). Mass(m/z): 369.2 [M+H]⁺.

### N-((10H-phenoxazin-3-yl)methyl)-4-(dimethylamino)-N-hydroxybutanamide (7)

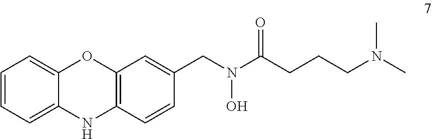 [SMILES: CN(C)CCCC(=O)N(O)Cc1ccc2c(c1)Oc1ccccc1N2]

The title compound 7 (4.0 mg) was prepared in a total yield of 7.3% as a yellow solid from N-((10H-phenoxazin-3-yl)methyl)hydroxylamine (36 mg, 0.16 mmol), 4-(dimethylamino)butanoic acid. hydrochloride salt (26 mg, 0.16 mmol), DIEA (62 mg, 0.48 mmol) and DMT-MM (49 mg, 0.18 mmol) according to the procedure for compound 6. ¹H NMR (400 MHz, Methanol-d₄) δ 6.71-6.66 (m, 2H), 6.60-6.51 (m, 3H), 6.43-6.35 (m, 2H), 4.54 (s, 2H), 3.04 (t, J=7.6 Hz, 2H), 2.80 (s, 6H), 2.64 (t, J=6.8 Hz, 2H), 2.03-1.94 (m, 2H). Mass(m/z): 342.2 [M+H]⁺.

### 2-(4-methylpiperazin-1-yl)-N-((8-(trifluoromethyl)-10H-phenoxazin-3-yl)methyl)acetamide (8)

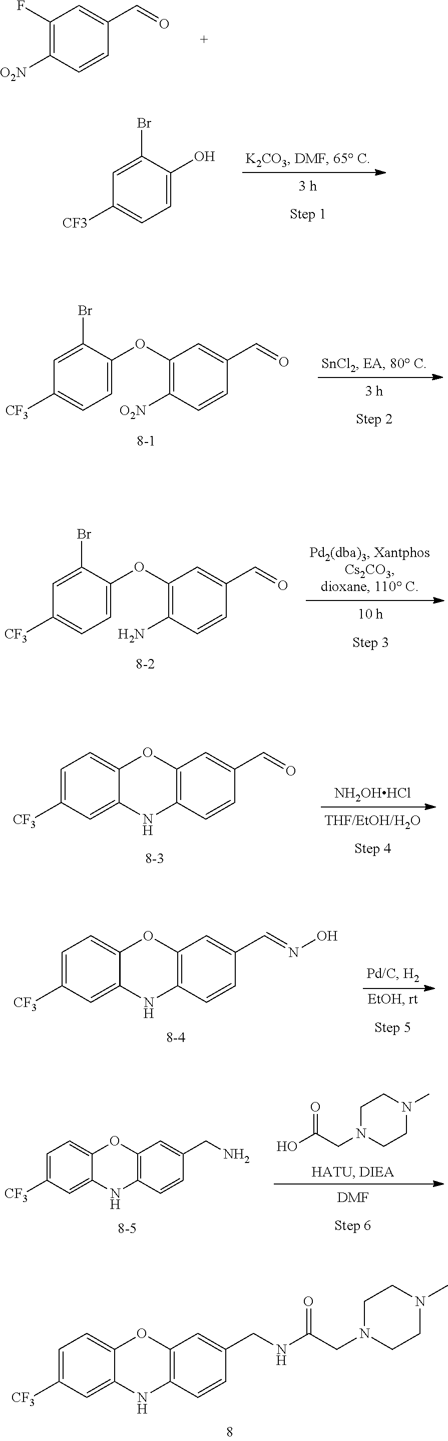

Step 1. A mixture of 3-fluoro-4-nitrobenzaldehyde (5 g, 29.6 mmol), 2-bromo-4-(trifluoromethyl)phenol (7.88 g, 32.5 mmol), dipotassium carbonate (8.18 g, 59.2 mmol and DMF (20 mL) was stirred at 65° C. for 3 h. The mixture was diluted with EA (400 mL) and washed with water (300 mL×3). The organic phase was concentrated and purified by flash, eluting with PE: EA=10:1 to 1:1 to give the desired product as a yellow oil (6 g, 46.6%).

Step 2. To a solution of 3-(2-bromo-4-(trifluoromethyl)phenoxy)-4-nitrobenzaldehyde (6 g, 15.3 mmol) and SnCl₂ (11.7 g, 61.2 mmol) in EA (30 mL) was stirred at 80° C. for 3 h. The mixture was diluted with EA (400 mL) and wash with water (100×2). The organic phase was concentrated and purified by flash, eluting with PE: EA=10:1 to 1:1 to give the desired product as a yellow solid (3 g, 49%). Mass(m/z): 359.7 [M+H]⁺.

Step 3. A mixture of 4-amino-3-(2-bromo-4-(trifluoromethyl)phenoxy)benzaldehyde (3 g, 7.5 mmol), Pd₂(dba)₃ (686 mg, 0.75 mmol), Xantphos (868 mg, 1.5 mmol), Cs₂CO₃ (4.89 g, 15 mmol) and Tol (30 mL) was stirred at 110° C. under N₂ atmosphere for 10 h. The mixture was concentrated and purified by flash eluting with PE: EA=10:1 to 1:1 to give the desired product as yellow solid (864 mg, 36%). ¹H NMR (400 MHz, DMSO-d₆) δ 9.63 (s, 1H), 9.30 (s, 1H), 7.37 (dd, J=8.0, 1.8 Hz, 1H), 7.05 (d, J=1.4 Hz, 1H), 7.00 (dd, J=8.2, 1.4 Hz, 1H), 6.82 (d, J=8.2 Hz, 1H), 6.73 (d, J=2.0 Hz, 1H), 6.59 (d, J=8.0 Hz, 1H). Mass(m/z): 280.0 [M+H]⁺. Step 4. To a solution of 8-(trifluoromethyl)-10H-phenoxazine-3-carbaldehyde (419 mg, 1.5 mmol) in EtOH (20 mL) was added Hydroxylamine hydrochloride (155 mg, 2.25 mmol). Then the reaction was stirred overnight at rt. The reaction mixture was concentrated under vacuum. The crude was used directly at next step. (100%). Mass(m/z): 295.1 [M+H]⁺.

Step 5. To a solution of (E)-8-(trifluoromethyl)-10H-phenoxazine-3-carbaldehyde oxime (441 mg, 1.5 mmol) in EtOH (20 mL) was added 10% Pd/C (16 mg, 0.015 ml) and AcOH (0.5 mL). Then the reaction was stirred overnight at rt under an atmosphere of Hydrogen. Pd/C was filtrated out. The PH of the filtration was adjusted to 8-9 with sodium carbonate solution. Then the mixture was extracted by DCM (20 mL×3). The combined organic layers were washed with brine (20 mL×3), dried over Na₂SO₄ and concentrated to give the desired product as yellow solid. (320 mg, 76.1%). 264.1 [M−NH2]⁺.

Step 6. The title compound 8 (9.5 mg) was prepared in a total yield of 22.6% as a yellow solid from (8-(trifluoromethyl)-10H-phenoxazin-3-yl)methanamine (28 mg, 0.1 mmol), 2-(4-methylpiperazin-1-yl)acetic acid (17 mg, 0.11 mmol), DIEA (39 mg, 0.3 mmol) and HATU (42 mg, 0.11 mmol) according to the procedure for compound 4. ¹H NMR (400 MHz, Methanol-d₄) δ 6.85-6.81 (m, 1H), 6.70-6.66 (m, 1H), 6.66-6.62 (m, 1H), 6.59 (dd, J=15.6, 2.0 Hz, 2H), 6.37 (d, J=8.0 Hz, 1H), 4.20 (s, 2H), 3.14 (s, 2H), 3.08-2.97 (m, 4H), 2.78-2.68 (m, 4H), 2.65 (s, 3H). Mass(m/z):421.3[M+H]⁺.

### 2-(4-methyl-2-oxopiperazin-1-yl)-N-((7-(trifluoromethyl)-10H-phenoxazin-3-yl)methyl)acetamide (9)

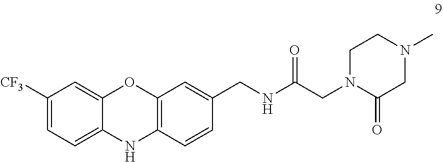 [SMILES: CN1CCN(CC(=O)NCc2ccc3c(c2)Oc2cc(C(F)(F)F)ccc2N3)C(=O)C1]

The title compound 9 (10.5 mg) was prepared in a total yield of 24.1% as a yellow solid from (7-(trifluoromethyl)-10H-phenoxazin-3-yl)methanamine (28 mg, 0.1 mmol), 2-(4-methyl-2-oxopiperazin-1-yl)acetic acid hydrochloride (25 mg, 0.12 mmol), DIEA (64.5 mg, 0.3 mmol) and HATU (38 mg, 0.1 mmol) according to the procedure for 4. ¹H NMR (400 MHz, Methanol-d4) δ 7.00-6.94 (m, 1H), 6.75 (d, J=2.0 Hz, 1H), 6.69-6.65 (m, 1H), 6.56 (d, J=1.9 Hz, 1H), 6.46 (d, J=8.2 Hz, 1H), 6.38 (dd, J=7.9, 0.8 Hz, 1H), 4.18 (s, 2H), 4.07 (s, 2H), 3.50-3.43 (m, 2H), 3.18 (s, 2H), 2.84-2.75 (m, 2H), 2.39 (s, 3H). Mass(m/z): 435.2[M+H]⁺.

### 2-(4-methyl-3-oxopiperazin-1-yl)-N-((2-methyl-7-(trifluoromethyl)-10H-phenoxazin-3-yl)methy)acetamide (10)

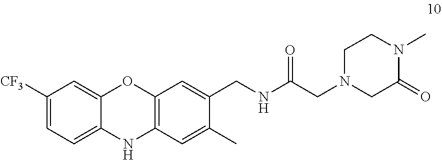 [SMILES: Cc1cc2c(cc1CNC(=O)CN1CCN(C)C(=O)C1)Oc1cc(C(F)(F)F)ccc1N2]

The title compound 10 (34.2 mg) was prepared in a total yield of 48.2% as a yellow solid from (2-methyl-7-(trifluoromethyl)-10H-phenoxazin-3-yl)methanamine (44 mg, 0.15 mmol), 2-(4-methyl-3-oxopiperazin-1-yl)acetic acid hydrochloride (37 mg, 0.18 mmol), DIEA (58 mg, 0.45 mmol) and HATU (68 mg, 0.18 mmol) according to the procedure for 4. ¹H NMR (400 MHz, Methanol-d₄) δ 6.96 (d, J=8.4 Hz, 1H), 6.73 (s, 1H), 6.51 (s, 1H), 6.45 (d, J=8.4 Hz, 1H), 6.26 (s, 1H), 4.21 (s, 2H), 3.40 (d, J=6.0 Hz, 2H), 3.21 (s, 2H), 3.16 (s, 2H), 2.94 (s, 3H), 2.80 (d, J=6.0 Hz, 2H), 2.13 (s, 3H). Mass(m/z): 449.3[M+H]⁺.

### 1-methyl-6-oxo-N-((7-(trifluoromethyl)-10H-phenoxazin-3-yl)methyl)piperidine-3-carboxamide (11)

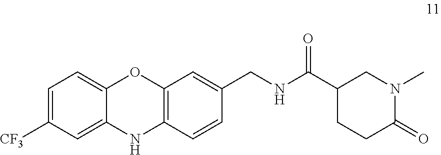 [SMILES: CN1CC(C(=O)NCc2ccc3c(c2)Oc2ccc(C(F)(F)F)cc2N3)CCC1=O]

The title compound 11 (21.8 mg) was prepared in a total yield of 34.6% as a yellow solid from (7-(trifluoromethyl)-10H-phenoxazin-3-yl)methanamine (44 mg, 0.15 mmol), 1-methyl-6-oxopiperidine-3-carboxylic acid (24 mg, 0.15 mmol), DIEA (58 mg, 0.45 mmol) and HATU (57 mg, 0.15 mmol) according to the procedure for 4. ¹H NMR (400 MHz, Methanol-d4) δ 6.97 (d, J=8.3 Hz, 1H), 6.75 (s, 1H), 6.67 (d, J=8.0 Hz, 1H), 6.55 (s, 1H), 6.43 (dd, J=30.2, 8.0 Hz, 2H), 4.16 (s, 2H), 3.57-3.37 (m, 2H), 2.94 (s, 3H), 2.84-2.74 (m, 1H), 2.49-2.28 (m, 2H), 2.06-1.91 (m, 2H). Mass(m/z): 420.2[M+H]⁺.

### 1-methyl-2-oxo-N-((7-(trifluoromethyl)-10H-phenoxazin-3-yl)methyl)piperidine-4-carboxamide (12)

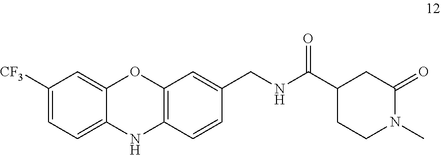 [SMILES: CN1CCC(C(=O)NCc2ccc3c(c2)Oc2cc(C(F)(F)F)ccc2N3)CC1=O]

The title compound 12 (12.5 mg) was prepared in a total yield of 29.8% as a yellow solid from (7-(trifluoromethyl)-10H-phenoxazin-3-yl)methanamine (28 mg, 0.1 mmol), 1-methyl-2-oxopiperidine-4-carboxylic acid (19 mg, 0.12 mmol), DIEA (39 mg, 0.3 mmol) and HATU (46 mg, 0.12 mmol) according to the procedure for compound 4. ¹H NMR (400 MHz, Methanol-d₄) δ 6.99-0.93 (m, 1H), 6.75 (d, J=2.0 Hz, 1H), 6.66 (dd, J=7.8, 1.8 Hz, 1H), 6.54 (d, J=1.8 Hz, 1H), 6.46 (dd, J=8.2, 1.0 Hz, 1H), 6.38 (d, J=7.8 Hz, 1H), 4.15 (d, J=2.8 Hz, 2H), 3.37 (dd, J=7.8, 4.6 Hz, 2H), 2.92 (s, 3H), 2.80-2.71 (m, 1H), 2.56-2.41 (m, 2H), 2.06-1.88 (m, 2H). Mass(m/z): 420.2 [M+H]⁺.

### 1-methyl-5-oxo-N-((7-(trifluoromethyl)-10H-phenoxazin-3-yl)methyl) pyrrolidine-3-carboxamide (13)

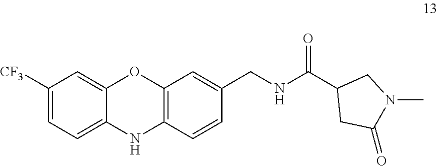 [SMILES: CN1CC(C(=O)NCc2ccc3c(c2)Oc2cc(C(F)(F)F)ccc2N3)CC1=O]

The title compound 13 (21.8 mg) was prepared in a total yield of 35.9% as a yellow solid from (7-(trifluoromethyl)-10H-phenoxazin-3-yl)methanamine (28 mg, 0.1 mmol), 1-methyl-5-oxopyrrolidine-3-carboxylic acid (22 mg, 0.15 mmol), DIEA (39 mg, 0.3 mmol) and HATU (46 mg, 0.12 mmol) according to the procedure for 4. ¹H NMR (400 MHz, Methanol-d₄) δ 6.98-6.95 (m, 1H), 6.75 (d, J=2.0 Hz, 1H), 6.67 (dd, J=8.0, 2.0 Hz, 1H), 6.55 (d, J=2.0 Hz, 1H), 6.46 (d, J=8.2 Hz, 1H), 6.39 (d, J=8.0 Hz, 1H), 4.16 (s, 2H), 3.66-3.53 (m, 2H), 3.24-3.19 (m, 1H), 2.83 (s, 1H), 2.59 (d, J=8.6 Hz, 2H). Mass(m/z): 406.2[M+H]⁺.

### 1-methyl-N-((7-(trifluoromethyl)-10H-phenoxazin-3-yl)methyl)piperidine-4-carboxamide (14)

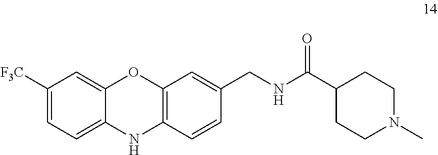 [SMILES: CN1CCC(C(=O)NCc2ccc3c(c2)Oc2cc(C(F)(F)F)ccc2N3)CC1]

The title compound 14 (11.7 mg) was prepared in a total yield of 28.9% (11.7 mg) as a yellow solid from (7-(trifluoromethyl)-10H-phenoxazin-3-yl)methanamine (28 mg, 0.1 mmol), 1-methylpiperidine-4-carboxylic acid (14 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 7.00-6.95 (m, 1H), 6.76-6.72 (m, 1H), 6.69-6.65 (m, 1H), 6.54 (d, J=2.0 Hz, 1H), 6.48-6.45 (m, 1H), 6.39 (d, J=7.8 Hz, 1H), 4.15 (s, 2H), 3.54-3.46 (m, 1H), 3.10-2.99 (m, 2H), 2.85 (s, 3H), 2.59-2.49 (m, 1H), 2.07-1.91 (m, 4H). Mass(m/z): 406.2 [M+H]⁺.

### 2-morpholino-N-((7-(trifluoromethyl)-10H-phenoxazin-3-yl)methyl)acetamide (15)

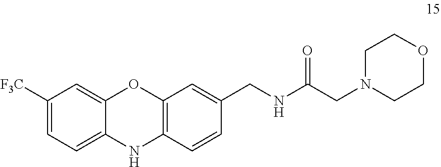 [SMILES: O=C(CN1CCOCC1)NCc1ccc2c(c1)Oc1cc(C(F)(F)F)ccc1N2]

The title compound 15 (29.7 mg) was prepared in a total yield of 72.9% as a yellow solid from (7-(trifluoromethyl)-10H-phenoxazin-3-yl)methanamine (28 mg, 0.1 mmol), 2-morpholinoacetic acid (14.5 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 7.00-6.95 (m, 1H), 6.75-6.72 (m, 1H), 6.71-6.67 (m, 1H), 6.59 (d, J=1.8 Hz, 1H), 6.49-6.45 (m, 1H), 6.39 (d, J=7.8 Hz, 1H), 4.22 (s, 2H), 3.99-3.89 (m, 6H), 3.37-3.32 (m, 2H). Mass(m/z): 408.2 [M+H]⁺.

### 4-(4-methylpiperazin-1-yl)-N-((7-(trifluoromethyl)-10H-phenoxazin-3-yl)methyl)butanamide 16

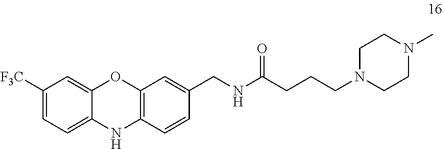 [SMILES: CN1CCN(CCCC(=O)NCc2ccc3c(c2)Oc2cc(C(F)(F)F)ccc2N3)CC1]

The title compound 16 (38.3 mg) was prepared in a total yield of 56.6% as a yellow solid from (7-(trifluoromethyl)-10H-phenoxazin-3-yl)methanamine (28 mg, 0.1 mmol), 4-(4-methylpiperazin-1-yl)butanoic acid (18.6 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 6.99-6.95 (m, 2H), 6.74 (d, J=2.0 Hz, 1H), 6.68 (dd, J=8.0, 2.0 Hz, 1H), 6.56 (d, J=2.0 Hz, 1H), 6.48-6.45 (m, 1H), 6.39 (d, J=8.0 Hz, 1H), 4.17 (s, 2H), 3.51-3.33 (m, 8H), 3.06 (t, J=7.0 Hz, 2H), 2.91 (s, 3H), 2.48-2.41 (m, 2H), 2.02-1.93 (m, 2H). Mass(m/z): 449.3[M+H]⁺.

### 2-(dimethylamino)-N-((7-(trifluoromethyl)-10H-phenoxazin-3-yl)methyl)acetamide (17)

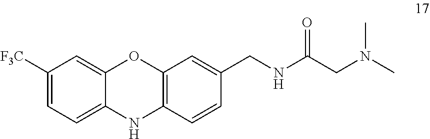 [SMILES: CN(C)CC(=O)NCc1ccc2c(c1)Oc1cc(C(F)(F)F)ccc1N2]

The title compound 17 (11.7 mg) was prepared in a total yield of 28.9% as a yellow solid from (7-(trifluoromethyl)-10H-phenoxazin-3-yl)methanamine (28 mg, 0.1 mmol), anddimethylglycine (10.3 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 7.00-6.94 (m, 1H), 6.76-6.71 (m, 1H), 6.71-6.66 (m, 1H), 6.57 (d, J=2.0 Hz, 1H), 6.46 (d, J=8.2 Hz, 1H), 6.42-6.37 (m, 1H), 4.21 (s, 2H), 3.47 (d, J=3.0 Hz, 2H), 2.61 (d, J=1.8 Hz, 6H).

Mass(m/z): 366.2 [M+H]⁺.

### 2-(pyrrolidin-1-yl)-N-((7-(trifluoromethyl)-10H-phenoxazin-3-yl)methyl)acetamide (18)

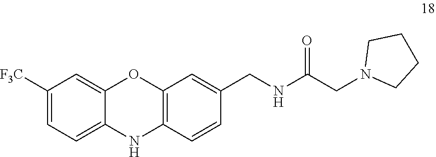 [SMILES: O=C(CN1CCCC1)NCc1ccc2c(c1)Oc1cc(C(F)(F)F)ccc1N2]

The title compound 18 (12.8 mg) was prepared in a total yield of 12.8% as a yellow solid from (7-(trifluoromethyl)-10H-phenoxazin-3-yl)methanamine (28 mg, 0.1 mmol), 2-(pyrrolidin-1-yl)acetic acid (12.9 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 7.00-6.94 (m, 1H), 6.75-6.73 (m, 1H), 6.71-6.67 (m, 1H), 6.58 (d, J=2.0 Hz, 1H), 6.49-6.45 (m, 4H), 6.40 (d, J=8.0 Hz, 1H), 4.21 (s, 2H), 3.79 (s, 2H), 3.21-3.15 (m, 4H), 2.03-1.99 (m, 4H).

Mass(m/z): 392.2 [M+H]⁺.

### 2-(1-methylpiperidin-4-yl)-N-((7-(trifluoromethyl)-10H-phenoxazin-3-yl)methyl)acetamide (19)

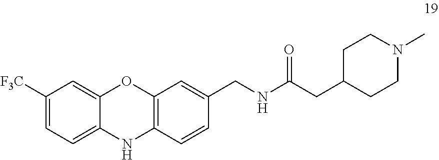 [SMILES: CN1CCC(CC(=O)NCc2ccc3c(c2)Oc2cc(C(F)(F)F)ccc2N3)CC1]

The title compound 19 (9.1 mg) was prepared in a total yield of 22.0% as a yellow solid from (7-(trifluoromethyl)-10H-phenoxazin-3-yl)methanamine (28 mg, 0.1 mmol), 2-(1-methylpiperidin-4-yl)acetic acid (15.7 mg, 0.1 mmol) DIEA (39 mg, 0.3 mmol) and HATU (38.0 mg, 0.1 mmol) according to the procedure for compound 4. ¹H NMR (400 MHz, Methanol-d₄) δ 7.00-6.95 (m, 1H), 6.76-6.73 (m, 1H), 6.69-6.65 (m, 1H), 6.55 (d, J=1.8 Hz, 1H), 6.48-6.45 (m, 1H), 6.39 (d, J=7.8 Hz, 1H), 4.15 (s, 2H), 3.51-3.39 (m, 2H), 3.06-2.94 (m, 2H), 2.83 (s, 3H), 2.23 (d, J=7.0 Hz, 2H), 2.10-1.92 (m, 3H), 1.60-1.46 (m, 2H). Mass(m/z): 420.3 [M+H]⁺.

### N-((7-(trifluoromethyl)-10H-phenoxazin-3-yl)methyl)quinuclidine-4-carboxamide (20)

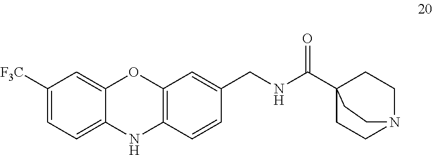 [SMILES: O=C(NCc1ccc2c(c1)Oc1cc(C(F)(F)F)ccc1N2)C12CCN(CC1)CC2]

The title compound 20 (4.9 mg) was prepared in a total yield of 9.2% as a yellow solid from (7-(trifluoromethyl)-10H-phenoxazin-3-yl)methanamine (28 mg, 0.1 mmol), 2-(1-methylpiperidin-4-yl)acetic acid (15.7 mg, 0.1 mmol), DIEA (39 mg, 0.3 mmol) and HATU (38.0 mg, 0.1 mmol) according to the procedure for 4. ¹H NMR (400 MHz, Methanol-d₄) δ 6.99-6.95 (m, 1H), 6.75-6.72 (m, 1H), 6.69-6.64 (m, 1H), 6.53 (d, J=2.0 Hz, 1H), 6.48-6.45 (m, 1H), 6.38 (d, J=8.0 Hz, 1H), 4.18 (s, 2H), 3.42-3.38 (m, 6H), 2.14-2.10 (m, 6H). Mass(m/z): 418.2 [M+H]⁺.

### 3-(4-methylpiperazin-1-yl)-N-((7-(trifluoromethyl)-10H-phenoxazin-3-yl)methyl)propenamide (21)

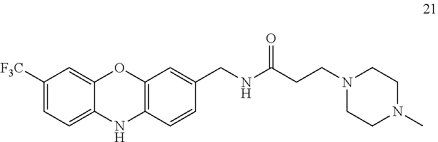 [SMILES: CN1CCN(CCC(=O)NCc2ccc3c(c2)Oc2cc(C(F)(F)F)ccc2N3)CC1]

The title compound 21 (22.7 mg) was prepared in a total yield of 34.3% as a yellow solid from (7-(trifluoromethyl)-10H-phenoxazin-3-yl)methanamine (28 mg, 0.1 mmol), 2-(pyrrolidin-1-yl)acetic acid (17.2 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 7.02-6.92 (m, 1H), 6.79-6.64 (m, 2H), 6.61-6.55 (m, 1H), 6.50-6.35 (m, 2H), 4.17 (s, 2H), 3.42-3.32 (m, 4H), 3.18-3.01 (m, 6H), 2.88 (s, 3H), 2.56 (t, J=6.8 Hz, 2H). 435.3 [M+H]⁺.

### 5-(dimethylamino)-N-((7-(trifluoromethyl)-10H-phenoxazin-3-yl)methyl)pentanamide (22)

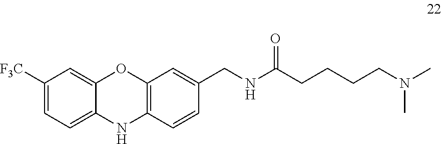 [SMILES: CN(C)CCCCC(=O)NCc1ccc2c(c1)Oc1cc(C(F)(F)F)ccc1N2]

The title compound 22 (3.7 mg) was prepared in a total yield of 9.1% as a yellow solid from (7-(trifluoromethyl)-10H-phenoxazin-3-yl)methanamine (28 mg, 0.1 mmol), 5-(dimethylamino)pentanoic acid (14.5 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 6.99-6.95 (m, 1H), 6.75 (d, J=2.0 Hz, 1H), 6.68 (dd, J=7.8, 2.0 Hz, 1H), 6.56 (d, J=2.0 Hz, 1H), 6.48-6.45 (m, 1H), 6.39 (d, J=7.8 Hz, 1H), 4.15 (s, 2H), 3.08 (t, J=7.2 Hz, 2H), 2.83 (s, 6H), 2.34-2.28 (m, 2H), 1.76-1.65 (m, 4H). 408.2 [M+H]⁺.

### 4-oxo-4-(((7-(trifluoromethyl)-10H-phenoxazin-3-yl)methyl)amino)butanoic acid (23)

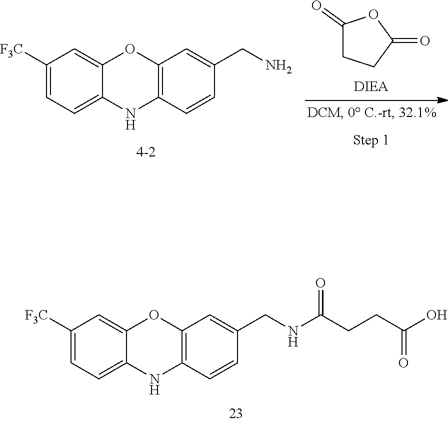 [SMILES: NCc1ccc2c(c1)Oc1cc(C(F)(F)F)ccc1N2 ; O=C1CCC(=O)O1 ; O=C(O)CCC(=O)NCc1ccc2c(c1)Oc1cc(C(F)(F)F)ccc1N2]

To a solution of (7-(trifluoromethyl)-10H-phenoxazin-3-yl)methanamine (28 mg, 0.1 mmol) and DIEA (39 mg, 0.3 mmol) in DCM (2 ml) was added dihydrofuran-2,5-dione (12 mg, 0.12 mmol) at 0° C. Then the reaction was stirred for 2 hours at rt. The reaction solution was concentrated and purified by prep-TLC (MeOH/DCM=1/10) to give the desired product as white solid (12.2 mg, 32.1%). ¹H NMR (400 MHz, Methanol-d₄) δ 6.96 (d, J=8.2 Hz, 1H), 6.74 (s, 1H), 6.66 (d, J=8.0 Hz, 1H), 6.55 (s, 1H), 6.45 (d, J=8.2 Hz, 1H), 6.37 (d, J=8.0 Hz, 1H), 4.15 (s, 2H), 2.61 (t, J=6.8 Hz, 2H), 2.49 (t, J=7.0 Hz, 2H). Mass(m/z): 381.1 [M+H]⁺.

### 1-methyl-2-oxo-N-((7-(trifluoromethyl)-10H-phenoxazin-3-yl)methyl)-1,2-dihydropyridine-4-carboxamide (24)

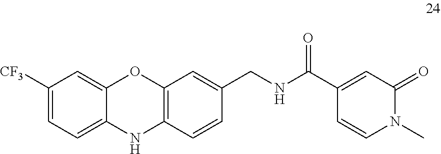 [SMILES: Cn1ccc(C(=O)NCc2ccc3c(c2)Oc2cc(C(F)(F)F)ccc2N3)cc1=O]

The title compound 24 (28.2 mg) was prepared in a total yield of 45.3% as a yellow solid from (7-(trifluoromethyl)-10H-phenoxazin-3-yl)methanamine (44 mg, 0.15 mmol), 1-methyl-2-oxo-1,2-dihydropyridine-4-carboxylic acid (23 mg, 0.15 mmol), DIEA (58 mg, 0.45 mmol) and HATU (57 mg, 0.15 mmol) according to the procedure for 4. ¹H NMR (400 MHz, Methanol-d₄) δ 7.73 (d, J=7.0 Hz, 1H), 6.98-6.94 (m, 1H), 6.89 (dd, J=2.0, 0.7 Hz, 1H), 6.75 (d, J=1.8 Hz, 1H), 6.72 (dd, J=8.0, 2.0 Hz, 1H), 6.67 (dd, J=7.0, 2.0 Hz, 1H), 6.60 (d, J=2.0 Hz, 1H), 6.47-6.44 (m, 1H), 6.40 (d, J=8.0 Hz, 1H), 4.31 (s, 2H), 3.58 (s, 3H). Mass(m/z): 416.2[M+H]⁺.

### N-hydroxy-N-((7-(trifluoromethyl)-10H-phenoxazin-3-yl)methyl)pivalamide (25)

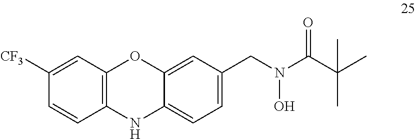 [SMILES: CC(C)(C)C(=O)N(O)Cc1ccc2c(c1)Oc1cc(C(F)(F)F)ccc1N2]

The title compound 25 (24.2 mg) was prepared in a total yield of 62.8% as a white solid from N-((7-(trifluoromethyl)-10H-phenoxazin-3-yl)methyl)hydroxylamine (30 mg, 0.101 mmol) and pivaloyl chloride (17 mg, 0.122 mmol) according to the procedure for compound 1. ¹H NMR (400 MHz, Methanol-d₄) δ 6.95 (d, J=7.4 Hz, 1H), 6.75 (d, J=1.8 Hz, 1H), 6.70-6.63 (m, 1H), 6.55 (d, J=1.8 Hz, 1H), 6.45 (d, J=8.2 Hz, 1H), 6.38 (d, J=7.8 Hz, 1H), 4.53 (s, 2H), 1.27 (s, 9H). Mass(m/z): 378.9 [M−H]⁺.

### N-hydroxy-N-((7-(trifluoromethyl)-10H-phenoxazin-3-yl)methyl)cyclohexanecarboxamide (26)

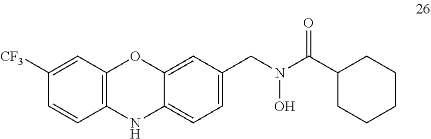 [SMILES: O=C(C1CCCCC1)N(O)Cc1ccc2c(c1)Oc1cc(C(F)(F)F)ccc1N2]

The title compound 26 (11.5 mg) was prepared in a total yield of 28.0% as a white solid from N-((7-(trifluoromethyl)-10H-phenoxazin-3-yl)methyl)hydroxylamine (30 mg, 0.101 mmol) and cyclohexanecarbonyl chloride (18 mg, 0.122 mmol) according to the procedure for compound 1. ¹H NMR (400 MHz, Methanol-d₄) δ 7.00-6.93 (m, 1H), 6.76 (d, J=2.0 Hz, 1H), 6.68 (dd, J=7.8, 1.8 Hz, 1H), 6.55 (d, J=1.8 Hz, 1H), 6.46 (d, J=8.2 Hz, 1H), 6.39 (d, J=7.8 Hz, 1H), 4.53 (s, 2H), 1.84-1.72 (m, 4H), 1.45-1.28 (m, 6H). Mass(m/z): 404.9 [M−H]⁺.

### N-hydroxy-2-(4-methylpiperazin-1-yl)-N-((7-(trifluoromethyl)-10H-phenoxazin-3-yl)methyl)acetamide (27)

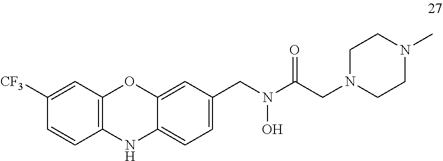 [SMILES: CN1CCN(CC(=O)N(O)Cc2ccc3c(c2)Oc2cc(C(F)(F)F)ccc2N3)CC1]

The title compound 27 (27.8 mg) was prepared in a total yield of 62.9% as a white solid from N-((7-(trifluoromethyl)-10H-phenoxazin-3-yl)methyl)hydroxylamine (30 mg, 0.101 mmol) and 2-(4-methylpiperazin-1-yl)acetic acid (21 mg, 0.132 mmol) according to the procedure for compound 2. ¹H NMR (400 MHz, Methanol-d₄) δ 6.96 (ddd, J=8.2, 2.0, 1.0 Hz, 1H), 6.73 (d, J=2.0 Hz, 1H), 6.70 (dd, J=8.0, 1.8 Hz, 1H), 6.57 (d, J=1.8 Hz, 1H), 6.51-6.45 (m, 1H), 6.40 (d, J=7.8 Hz, 1H), 4.53 (s, 2H), 3.47 (s, 2H), 2.80 (m, 8H), 2.52 (s, 3H). Mass(m/z): 437.2 [M+H]⁺.

### 1-ethyl-5-oxo-N-((8-(trifluoromethyl)-10H-phenoxazin-3-yl)methyl)pyrrolidine-3-carboxamide (28)

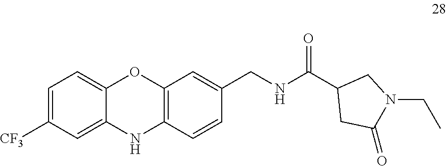 [SMILES: CCN1CC(C(=O)NCc2ccc3c(c2)Oc2ccc(C(F)(F)F)cc2N3)CC1=O]

The title compound 28 (26.6 mg) was prepared in a total yield of 59.1% as a white solid from (8-(trifluoromethyl)-10H-phenoxazin-3-yl)methanamine (30 mg, 0.107 mmol) and 1-ethyl-5-oxopyrrolidine-3-carboxylic acid (17 mg, 0.107 mmol) according to the procedure for 4. ¹H NMR (400 MHz, Methanol-d₄) δ 6.81 (dd, J=8.4, 2.1 Hz, 1H), 6.70-6.59 (m, 3H), 6.53 (d, J=2.0 Hz, 1H), 6.37 (d, J=8.0 Hz, 1H), 4.15 (s, 2H), 3.65 (dd, J=9.8, 8.8 Hz, 1H), 3.54 (dd, J=9.8, 6.0 Hz, 1H), 3.33 (d, J=8.0 Hz, 1H), 3.30 (s, 1H), 3.23 (qd, J=8.4, 6.0 Hz, 1H), 2.60 (d, J=8.4 Hz, 2H), 1.11 (t, J=7.2 Hz, 3H). Mass(m/z): 420.3[M+H]⁺.

### 1-methyl-6-oxo-N-((8-(trifluoromethyl)-10H-phenoxazin-3-yl)methyl)piperidine-3-carboxamide (29)

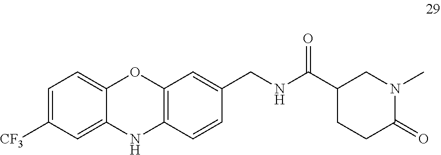 [SMILES: CN1CC(C(=O)NCc2ccc3c(c2)Oc2ccc(C(F)(F)F)cc2N3)CCC1=O]

The title compound 29 (20.7 mg) was prepared in a total yield of 43.4% as a white solid from (8-(trifluoromethyl)-10H-phenoxazin-3-yl)methanamine (30 mg, 0.107 mmol) and 1-methyl-6-oxopiperidine-3-carboxylic acid (17 mg, 0.107 mmol) according to the procedure for 4. ¹H NMR (400 MHz, Methanol-d₄) δ 6.82 (ddd, J=8.4, 2.2, 0.8 Hz, 1H), 6.70-6.59 (m, 3H), 6.54 (d, J=2.0 Hz, 1H), 6.38 (d, J=8.0 Hz, 1H), 4.15 (d, J=1.2 Hz, 2H), 3.53 (dd, J=12.4, 9.6 Hz, 1H), 3.42 (ddd, J=12.4, 5.6, 1.2 Hz, 1H), 2.93 (s, 3H), 2.81 (tdd, J=9.6, 5.6, 4.0 Hz, 1H), 2.38 (qt, J=11.4, 5.6 Hz, 2H), 2.05-1.93 (m, 2H). Mass(m/z): 420.3[M+H]⁺.

### N-((7-(trifluoromethyl)-10H-phenoxazin-3-yl)methyl)quinuclidine-3-carboxamide (30)

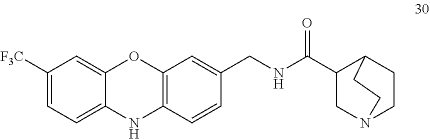 [SMILES: O=C(NCc1ccc2c(c1)Oc1cc(C(F)(F)F)ccc1N2)C1CN2CCC1CC2]

The title compound 30 (17.1 mg) was prepared in a total yield of 32.2% as a yellow solid from (7-(trifluoromethyl)-10H-phenoxazin-3-yl)methanamine (28 mg, 0.1 mmol), quinuclidine-3-carboxylic acid (15.5 mg, 0.1 mmol), DIEA (39 mg, 0.3 mmol) and HATU (38.0 mg, 0.1 mmol) according to the procedure for compound 4. ¹H NMR (400 MHz, DMSO-d₆) δ 9.27 (s, 1H), 8.70 (s, 1H), 8.51 (t, J=5.8 Hz, 1H), 7.08-7.00 (m, 1H), 6.84 (d, J=2.0 Hz, 1H), 6.64 (dd, J=7.8, 1.8 Hz, 1H), 6.54-6.50 (m, 2H), 6.41 (d, J=7.8 Hz, 1H), 4.14-4.03 (m, 2H), 3.60-3.52 (m, 1H), 3.27 (t, J=11.4 Hz, 1H), 3.23-3.12 (m, 4H), 2.91-2.84 (m, 1H), 2.27-2.21 (m, 1H), 1.87-1.76 (m, 2H), 1.70-1.61 (m, 2H). Mass(m/z): 418.2 [M+H]⁺.

### 1-methyl-2-oxo-N-((8-(trifluoromethyl)-10H-phenoxazin-3-yl)methyl)-1,2-dihydropyridine-4-carboxamide (31)

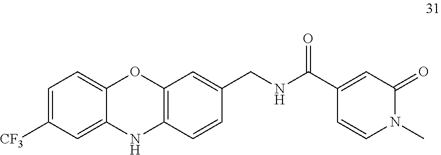 [SMILES: Cn1ccc(C(=O)NCc2ccc3c(c2)Oc2ccc(C(F)(F)F)cc2N3)cc1=O]

The title compound 31 (42.2 mg) was prepared in a total yield of 93.8% as a white solid from (8-(trifluoromethyl)-10H-phenoxazin-3-yl)methanamine (30 mg, 0.107 mmol) and 1-methyl-2-oxo-1,2-dihydropyridine-4-carboxylic acid (17 mg, 0.107 mmol) according to the procedure for 4. ¹H NMR (400 MHz, DMSO-d₆) δ 9.03 (t, J=6.0 Hz, 1H), 8.48 (s, 1H), 7.76 (d, J=7.0 Hz, 1H), 6.85 (ddt, J=8.2, 2.0, 0.8 Hz, 1H), 6.80 (d, J=2.0 Hz, 1H), 6.74-6.67 (m, 2H), 6.63 (d, J=2.2 Hz, 1H), 6.57 (d, J=2.0 Hz, 1H), 6.51 (dd, J=7.0, 2.0 Hz, 1H), 6.40 (dd, J=7.8, 1.2 Hz, 1H), 5.73 (d, J=0.4 Hz, 1H), 4.19 (d, J=5.8 Hz, 2H), 3.42 (s, 3H). Mass(m/z): 416.3[M+H]⁺.

### 4-methyl-N-((7-(trifluoromethyl)-10H-phenoxazin-3-yl)methyl)piperazine-1-carboxamide (32)

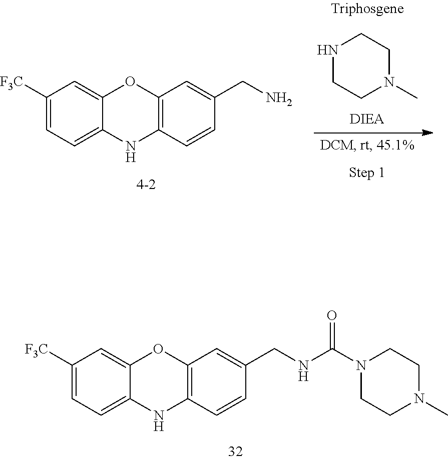

To a solution of (7-(trifluoromethyl)-10H-phenoxazin-3-yl)methanamine (28 mg, 0.1 mmol) in DCM (2 mL) was added triphosgene (29.6 mg, 0.11 mmol). Then the reaction mixture was stirred for 1 hour at. DIEA (39 mg, 0.3 mmol) and 1-methylpiperazine (11 mg, 0.11 mmol) were added, the reaction mixture was stirred for 3 hours at. The reaction solution was washed with water (3×5 mL), dried over Na₂SO₄ and concentrated under vacuum. The residue was purified by prep-TLC (MeOH/DCM=1/10) to give the desired product as yellow solid (18.3 mg, 45.1%). ¹H NMR (400 MHz, Methanol-d₄) δ 6.99-6.94 (m, 4H), 6.74 (d, J=2.0 Hz, 1H), 6.67 (dd, J=8.0, 2.0 Hz, 1H), 6.56 (d, J=1.8 Hz, 1H), 6.46 (dd, J=8.2, 0.8 Hz, 1H), 6.37 (d, J=7.8 Hz, 1H), 4.14 (s, 2H), 3.63-3.50 (m, 4H), 2.86 (t, J=4.6 Hz, 4H), 2.62 (s, 3H). Mass(m/z): 407.2 [M+H]⁺.

### 3-(dimethylamino)-N-((7-(trifluoromethyl)-10H-phenoxazin-3-yl)methyl)propenamide (33)

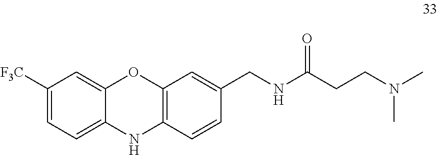 [SMILES: CN(C)CCC(=O)NCc1ccc2c(c1)Oc1cc(C(F)(F)F)ccc1N2]

The title compound 33 (16.3 mg) was prepared in a total yield of 43.1% as a yellow solid from (7-(trifluoromethyl)-10H-phenoxazin-3-yl)methanamine (28 mg, 0.1 mmol), 3-(dimethylamino)propanoic acid (11.7 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ6.99-6.94 (m, 1H), 6.73 (d, J=2.4 Hz, 1H), 6.70-6.66 (m, 1H), 6.57 (d, J=1.8 Hz, 1H), 6.49-6.43 (m, 1H), 6.39 (d, J=7.8 Hz, 1H), 4.17 (s, 2H), 3.44-3.36 (m, 2H), 2.89 (s, 6H), 2.74 (t, J=6.6 Hz, 2H). Mass(m/z): 380.2 [M+H]⁺.

### 4-(pyrrolidin-1-yl)-N-((7-(trifluoromethyl)-10H-phenoxazin-3-yl)methyl)butanamide (34)

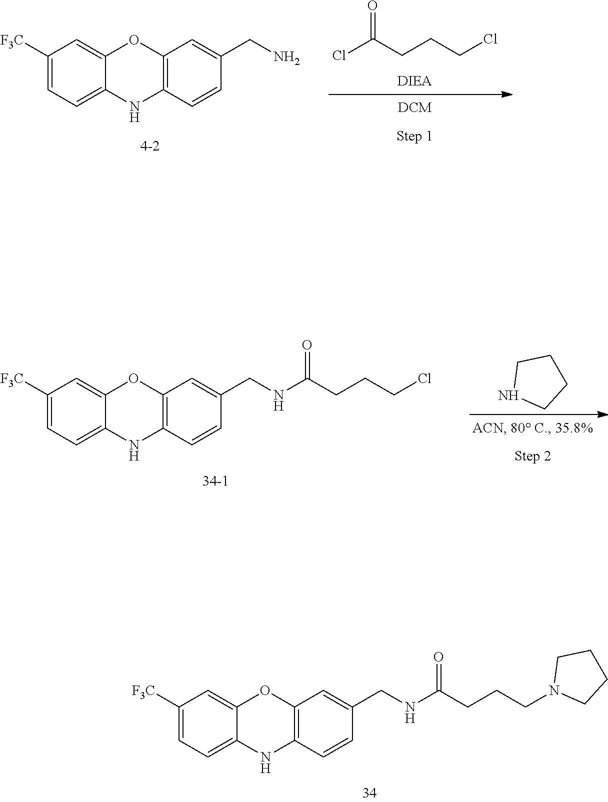 [SMILES: NCc1ccc2c(c1)Oc1cc(C(F)(F)F)ccc1N2 ; O=C(Cl)CCCCl ; O=C(CCCCl)NCc1ccc2c(c1)Oc1cc(C(F)(F)F)ccc1N2 ; C1CCNC1 ; O=C(CCCN1CCCC1)NCc1ccc2c(c1)Oc1cc(C(F)(F)F)ccc1N2]

Step 1. To a solution of (7-(trifluoromethyl)-10H-phenoxazin-3-yl)methanamine (28 mg, 0.1 mmol) and DIEA (39 mg, 0.3 mmol) in DCM (2 mL) was added dropwise 4-chlorobutanoyl chloride (14 mg, 0.1 mmol) at 0° C. Then the reaction was stirred for 2 hours at 0° C. The reaction solution was washed with water (3×5 mL), dried over Na₂SO₄ and concentrated under vacuum to afford the crude product as a yellow oil. The crude was used directly at next step.

Mass(m/z): 385.2[M+H]⁺

Step 2. To a solution of 34-1 (38.4 mg, 0.1 mmol) in ACN (2 ml) was added pyrrolidine (0.1 ml). Then the reaction was stirred overnight at 80° C. After cooling to rt. 10 ml of water was added. Then the mixture was extracted by DCM (10 mL×3). The combined organic layers were washed with water (15 mL×3), dried over Na₂SO₄ and concentrated under vacuum. The residue was purified by prep-TLC (MeOH/DCM=1/10) to give the desired product as yellow solid (15.0 mg, 35.8%). 1H NMR (400 MHz, Methanol-d₄) δ 6.99-6.95 (m, 1H), 6.74 (d, J=2.0 Hz, 1H), 6.68 (dd, J=8.0 Hz, 2.0 Hz, 1H), 6.56 (d, J=2.0 Hz, 1H), 6.50-6.45 (m, 1H), 6.40 (d, J=7.8 Hz, 1H), 4.15 (s, 2H), 3.35-3.31 (m, 2H), 3.23-3.17 (m, 2H), 2.39 (d, J=7.0 Hz, 2H), 2.10-1.99 (m, 6H). Mass(m/z): 420.2 [M+H]⁺

### 2-(4-(4-methylpiperazin-1-yl)piperidin-1-yl)-N-((7-(trifluoromethyl)-10H-phenoxazin-3-yl)methyl)acetamide (35)

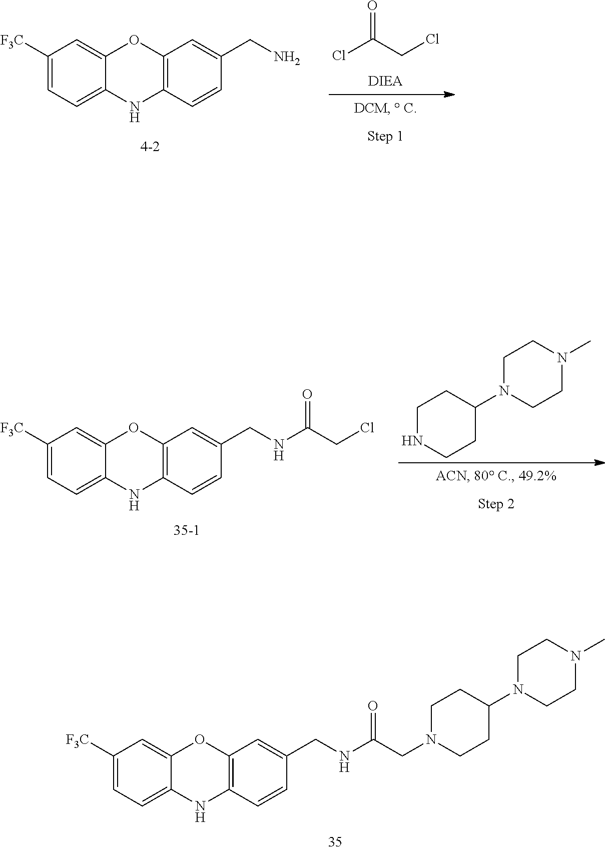 [SMILES: NCc1ccc2c(c1)Oc1cc(C(F)(F)F)ccc1N2 ; O=C(Cl)CCl ; O=C(CCl)NCc1ccc2c(c1)Oc1cc(C(F)(F)F)ccc1N2 ; CN1CCN(C2CCNCC2)CC1 ; CN1CCN(C2CCN(CC(=O)NCc3ccc4c(c3)Oc3cc(C(F)(F)F)ccc3N4)CC2)CC1]

Step 1. The title compound 35-1 (2.5 mg) was prepared in a total yield of 100% as a crude as a yellow solid from (7-(trifluoromethyl)-10H-phenoxazin-3-yl)methanamine (28 mg, 0.1 mmol), 2-chloroacetyl chloride (22.4 mg, 0.2 mmol) and DIEA (39 mg, 0.3 mmol) according to the procedure for 34-1. Mass(m/z): 356.0[M+H]⁺

Step 2. The title compound 35 (41.6 mg) was prepared in a total yield of 49.2% as a yellow solid from 2-chloro-N-((7-(trifluoromethyl)-10H-phenoxazin-3-yl)methyl)acetamide (35.6 mg, 0.1 mmol) and 1-methyl-4-(piperidin-4-yl)piperazine (91.5 mg, 0.5 mmol) according to the procedure for compound 34. ¹H NMR (400 MHz, Methanol-d₄) δ 6.98 (d, J=8.2 Hz, 1H), 6.74 (d, J=2.0 Hz, 1H), 6.69 (d, J=8.2 Hz, 1H), 6.57 (s, 1H), 6.47 (d, J=8.2 Hz, 1H), 6.39 (d, J=8.0 Hz, 1H), 4.21 (s, 2H), 3.68-3.58 (m, 3H), 3.30-2.96 (m, 12H), 2.87 (s, 3H), 2.35-2.20 (m, 2H), 1.97-1.81 (m, 2H). Mass(m/z): 504.3[M+H]⁺.

### 2-(4-(pyrrolidin-1-yl)piperidin-1-yl)-N-((7-(trifluoromethyl)-10H-phenoxazin-3-yl)methyl)acetamide (36)

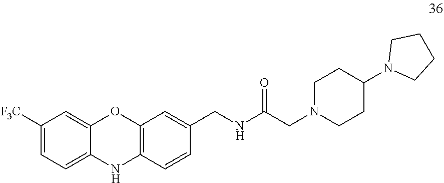 [SMILES: O=C(CN1CCC(N2CCCC2)CC1)NCc1ccc2c(c1)Oc1cc(C(F)(F)F)ccc1N2]

The title compound 36 (3.9 mg) was prepared in a total yield of 8.2% as a yellow solid from 2-chloro-N-((7-(trifluoromethyl)-10H-phenoxazin-3-yl)methyl)acetamide (35.6 mg, 0.1 mmol) and 4-(pyrrolidin-1-yl)piperidine (77.0 mg, 0.5 mmol) according to the procedure for compound 34. ¹H NMR (400 MHz, Methanol-d₄) δ 7.00-6.95 (m, 1H), 6.75 (d, J=2.0 Hz, 1H), 6.69 (dd, J=8.0, 1.8 Hz, 1H), 6.57 (d, J=1.8 Hz, 1H), 6.50-6.44 (m, 1H), 6.39 (d, J=7.8 Hz, 1H), 4.20 (s, 2H), 3.76-3.38 (m, 3H), 3.23-2.97 (m, 8H), 2.34 (t, J=12.2 Hz, 2H), 2.16-2.07 (m, 4H), 1.89-1.79 (m, 2H). Mass(m/z): 475.3[M+H]⁺.

### 2-(4-(3-(dimethylamino)propyl)piperazin-1-yl)-N-((7-(trifluoromethyl)-10H-phenoxazin-3-yl)methyl)acetamide (37)

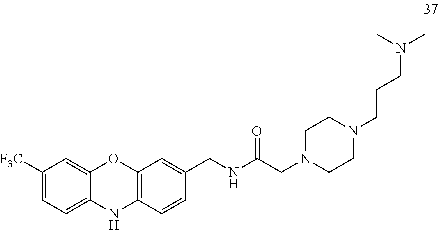 [SMILES: CN(C)CCCN1CCN(CC(=O)NCc2ccc3c(c2)Oc2cc(C(F)(F)F)ccc2N3)CC1]

The title compound 37 (11.6 mg) was prepared in a total yield of 23.6% as a yellow solid from 2-chloro-N-((7-(trifluoromethyl)-10H-phenoxazin-3-yl)methyl)acetamide (35.6 mg, 0.1 mmol) and N,N-dimethyl-3-(piperazin-1-yl)propan-1-amine (85.5 mg, 0.5 mmol) according to the procedure for compound 34. ¹H NMR (400 MHz, Methanol-d₄) δ 7.01-6.95 (m, 1H), 6.73 (d, J=2.0 Hz, 1H), 6.69 (dd, J=8.0, 1.8 Hz, 1H), 6.57 (d, J=1.8 Hz, 1H), 6.47 (d, J=8.2 Hz, 1H), 6.39 (d, J=7.8 Hz, 1H), 4.21 (s, 2H), 3.45 (s, 2H), 3.26-3.12 (m, 6H), 3.10-2.95 (m, 6H), 2.90 (s, 6H), 2.16-2.05 (m, 2H). Mass(m/z): 492.3[M+H]⁺

### 2-(4-methyl-1,4-diazepan-1-yl)-N-((7-(trifluoromethyl)-10H-phenoxazin-3-yl)methyl)acetamide (38)

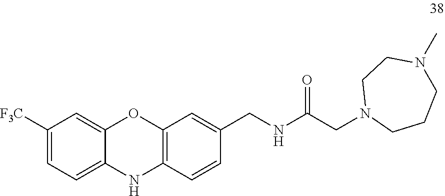 [SMILES: CN1CCCN(CC(=O)NCc2ccc3c(c2)Oc2cc(C(F)(F)F)ccc2N3)CC1]

The title compound 38 (11.3 mg) was prepared in a total yield of 26.0% as a yellow solid from 2-chloro-N-((7-(trifluoromethyl)-10H-phenoxazin-3-yl)methyl)acetamide (35.6 mg, 0.1 mmol) and 1-methyl-1,4-diazepane (57 mg, 0.5 mmol) according to the procedure for compound 34. ¹H NMR (400 MHz, Methanol-d₄) δ 6.99-6.95 (m, 2H), 6.75-6.68 (m, 2H), 6.59 (t, J=1.6 Hz, 1H), 6.47 (d, J=8.2 Hz, 1H), 6.39 (dd, J=8.0, 1.2 Hz, 1H), 4.22 (s, 2H), 3.49-3.39 (m, 2H), 3.36-3.31 (m, 2H), 3.02-2.78 (m, 7H), 2.10-1.95 (m, 2H). Mass(m/z): 435.2[M+H]⁺

### 2-(4-methyl-3-oxopiperazin-1-yl)-N-((7-(trifluoromethyl)-10H-phenoxazin-3-yl)methyl)acetamide (39)

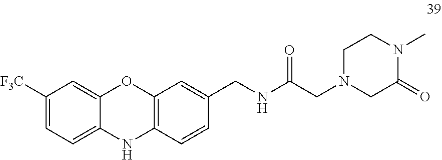 [SMILES: CN1CCN(CC(=O)NCc2ccc3c(c2)Oc2cc(C(F)(F)F)ccc2N3)CC1=O]

The title compound 39 (12.1 mg) was prepared in a total yield of 27.9% as a yellow solid from 2-chloro-N-((7-(trifluoromethyl)-10H-phenoxazin-3-yl)methyl)acetamide (35.6 mg, 0.1 mmol) and 1-methylpiperazin-2-one (57 mg, 0.5 mmol) according to the procedure for compound 34. ¹H NMR (400 MHz, Methanol-d₄) δ 6.99-6.94 (m, 1H), 6.75 (d, J=2.0 Hz, 1H), 6.70-6.66 (m, 1H), 6.56 (d, J=2.0 Hz, 1H), 6.46 (d, J=8.2 Hz, 1H), 6.38 (dd, J=8.0, 0.8 Hz, 1H), 4.19 (s, 2H), 3.43-3.38 (m, 2H), 3.20 (s, 2H), 3.16 (s, 2H), 2.94 (s, 3H), 2.83-2.76 (m, 2H).

Mass(m/z): 435.2[M+H]⁺

### 2-(3-(dimethylamino)piperidin-1-yl)-N-((7-(trifluoromethyl)-10H-phenoxazin-3-yl)methyl)acetamide (40)

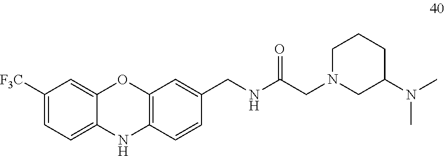 [SMILES: CN(C)C1CCCN(CC(=O)NCc2ccc3c(c2)Oc2cc(C(F)(F)F)ccc2N3)C1]

The title compound 40 (6.5 mg) was prepared in a total yield of 14.5% as a yellow solid from 2-chloro-N-((7-(trifluoromethyl)-10H-phenoxazin-3-yl)methyl)acetamide (35.6 mg, 0.1 mmol) and N,N-dimethylpiperidin-3-amine (64 mg, 0.5 mmol) according to the procedure for compound 34. ¹H NMR (400 MHz, Methanol-d₄) δ 6.99-6.95 (m, 1H), 6.75-6.68 (m, 2H), 6.59 (d, J=1.8 Hz, 1H), 6.49-6.44 (m, 1H), 6.39 (dd, J=8.0, 0.8 Hz, 1H), 4.21 (d, J=2.4 Hz, 2H), 3.37-3.33 (m, 1H), 3.25-3.11 (m, 2H), 2.93-2.86 (m, 7H), 2.80-2.74 (m, 1H), 2.66-2.57 (m, 1H), 2.54-2.46 (m, 1H), 1.93-1.67 (m, 4H). Mass(m/z): 449.3[M+H]⁺.

### 2-(4-acetylpiperazin-1-yl)-N-((7-(trifluoromethyl)-10H-phenoxazin-3-yl)methyl)acetamide (41)

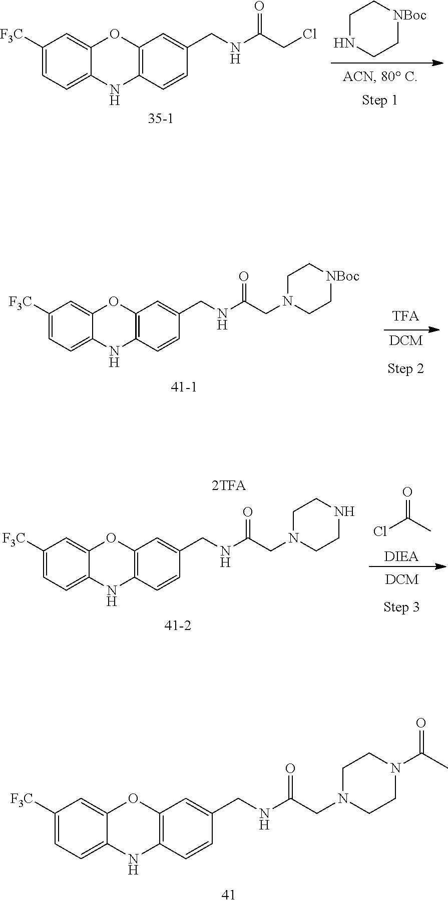

Step 1. The title compound 41-1 (101 mg) was prepared in a total yield of 100% as a yellow solid from 2-chloro-N-((7-(trifluoromethyl)-10H-phenoxazin-3-yl)methyl)acetamide (35.6 mg, 0.1 mmol) and tert-butyl piperazine-1-carboxylate (186 mg, 0.5 mmol) according to the procedure for 34. Mass(m/z): 507.3[M+H]⁺.

Step 2. To a solution of t compound 41-1 (101 mg, 2 mmol) in DCM (5 mL) was added TFA (3 mL). Then the reaction was stirred for 30 mins at rt. The reaction solution was concentrated under vacuum to afford a crude product as a yellow solid. The crude used directly at next step (89 mg, 69%). Mass(m/z): 407.2[M+H]⁺.

Step 3. To a solution of 41-2 (89 mg, 0.14 mmol) and DIEA (88.9 mg, 0.69 mmol) in DCM (5 ml) was added acetyl chloride (16.4 mg, 0.21 mmol) at 0° C. Then the reaction was stirred for 30 mins at rt. The reaction solution was concentrated and purified by prep-TLC (MeOH/DCM=1/10) to afford the desired product as a yellow solid. (22.4 mg, 36.5%). 1H NMR (400 MHz, Methanol-d₄) δ 6.99-6.94 (m, 1H), 6.75 (d, J=2.0 Hz, 1H), 6.70-6.67 (m, 1H), 6.57 (d, J=1.8 Hz, 1H), 6.48-6.44 (m, 1H), 6.38 (d, J=7.8 Hz, 1H), 4.21 (s, 2H), 3.66-3.55 (m, 4H), 3.08 (s, 2H), 2.51 (dt, J=20.2, 5.2 Hz, 4H), 2.08 (s, 3H). Mass(m/z): 449.3[M+H]⁺

### N-hydroxy-N-((7-(trifluoromethyl)-10H-phenoxazin-3-yl)methyl)benzamide (42)

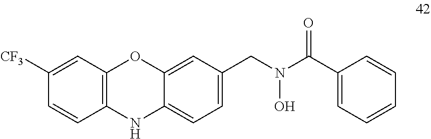 [SMILES: O=C(c1ccccc1)N(O)Cc1ccc2c(c1)Oc1cc(C(F)(F)F)ccc1N2]

The title compound 42 (22.6 mg) was prepared in a total yield of 55.8% as a white solid from N-((7-(trifluoromethyl)-10H-phenoxazin-3-yl)methyl)hydroxylamine (30 mg, 0.101 mmol) and benzoyl chloride (17 mg, 0.122 mmol) according to the procedure for compound 1. ¹H NMR (400 MHz, Methanol-d₄) δ 8.13-8.08 (m, 2H), 8.03-7.98 (m, 2H), 7.74-7.65 (m, 1H), 7.58-7.50 (m, 4H), 7.45-7.39 (m, 2H), 5.02 (s, 2H). Mass(m/z): 398.9 [M−H]⁺

### N-((7-methyl-10H-phenoxazin-3-yl)methyl)-2-(4-methylpiperazin-1-yl)acetamide (43)

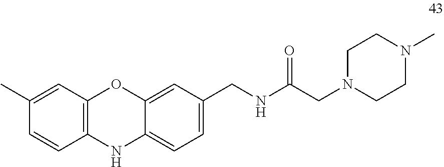

The title compound 43 (36.2 mg) was prepared in a total yield of 74.5% as a white solid from (7-methyl-10H-phenoxazin-3-yl)methanamine (30 mg, 0.133 mmol) and 2-(4-methylpiperazin-1-yl)acetic acid (27 mg, 0.173 mmol) according to the procedure for compound 2. ¹H NMR (400 MHz, Methanol-d₄) δ 6.65-6.60 (m, 1H), 6.52 (d, J=2.0 Hz, 1H), 6.50 (ddd, J=7.9, 2.0, 0.8 Hz, 1H), 6.38 (dd, J=2.0, 0.8 Hz, 1H), 6.35 (d, J=7.8 Hz, 1H), 6.30 (d, J=7.8 Hz, 1H), 4.18 (s, 2H), 3.12 (s, 2H), 3.00 (m, 4H), 2.71 (m, 4H), 2.63 (s, 3H), 2.11 (s, 3H).

Mass(m/z): 367.3 [M+H]⁺.

### 1-isopropyl-N-((7-methyl-10H-phenoxazin-3-yl)methyl)piperidine-4-carboxamide (44)

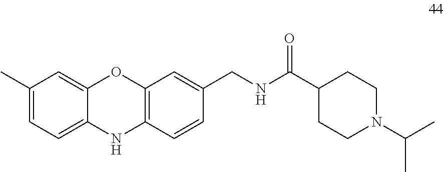 [SMILES: Cc1ccc2c(c1)Oc1cc(CNC(=O)C3CCN(C(C)C)CC3)ccc1N2]

The title compound 44 (37.6 mg) was prepared in a total yield of 75.2% as a white solid from (7-methyl-10H-phenoxazin-3-yl)methanamine (30 mg, 0.133 mmol) and 1-isopropylpiperidine-4-carboxylic acid hydrochloride (36 mg, 0.173 mmol) according to the procedure for compound 2. ¹H NMR (400 MHz, Methanol-d₄) δ 6.62 (dd, J=7.8, 2.0 Hz, 1H), 6.52-6.48 (m, 2H), 6.38 (dd, J=1.8, 0.8 Hz, 1H), 6.35 (d, J=7.8 Hz, 1H), 6.30 (d, J=7.8 Hz, 1H), 4.14 (s, 2H), 3.49 (m, 3H), 3.10 (m, 2H), 2.58 (m, 1H), 2.12 (s, 3H), 2.11-1.88 (m, 4H), 1.35 (d, J=6.8 Hz, 6H). Mass(m/z): 380.3 [M+H]⁺.

### 1-isopropyl-N-((8-(trifluoromethyl)-10H-phenoxazin-3-yl)methyl)piperidine-4-carboxamide (45)

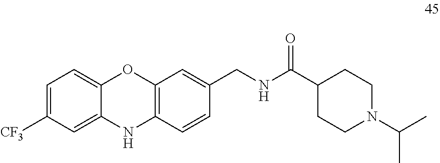 [SMILES: CC(C)N1CCC(C(=O)NCc2ccc3c(c2)Oc2ccc(C(F)(F)F)cc2N3)CC1]

The title compound 45 (12.3 mg) was prepared in a total yield of 28.4% as a yellow solid from (8-(trifluoromethyl)-10H-phenoxazin-3-yl)methanamine (28 mg, 0.1 mmol), isopropylpiperidine-4-carboxylic acid hydrochloride (23 mg, 0.11 mmol), DIEA (39 mg, 0.3 mmol) and HATU (42 mg, 0.11 mmol) according to the procedure for compound 4. ¹H NMR (400 MHz, Methanol-d₄) δ 6.86-6.81 (m, 1H), 6.69-6.60 (m, 3H), 6.54 (d, J=1.8 Hz, 1H), 6.37 (d, J=7.8 Hz, 1H), 4.15 (s, 2H), 3.53-3.46 (m, 3H), 3.15-2.97 (m, 2H), 2.60-2.51 (m, 1H), 2.14-1.94 (m, 4H), 1.35 (d, J=6.8, 6H). Mass(m/z):434.3 [M+H]⁺.

### 2-(pyrazin-2-yl)-N-((7-(trifluoromethyl)-10H-phenoxazin-3-yl)methyl)acetamide (46)

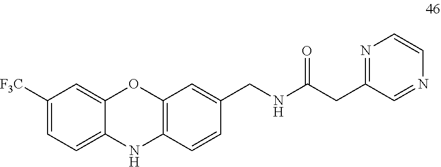 [SMILES: O=C(Cc1cnccn1)NCc1ccc2c(c1)Oc1cc(C(F)(F)F)ccc1N2]

The title compound 46 (2.5 mg) was prepared in a total yield of 6.3% as a yellow solid from (7-(trifluoromethyl)-10H-phenoxazin-3-yl)methanamine (28 mg, 0.1 mmol), 2-(pyrazin-2-yl)acetic acid (17 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 8.61 (d, J=1.6 Hz, 1H), 8.57 (dd, J=2.8, 1.6 Hz, 1H), 8.49 (d, J=2.6 Hz, 1H), 6.99-6.92 (m, 1H), 6.76 (d, J=2.0 Hz, 1H), 6.68 (dd, J=7.8, 1.8 Hz, 1H), 6.57 (d, J=1.8 Hz, 1H), 6.46 (d, J=8.2 Hz, 1H), 6.38 (d, J=7.8 Hz, 1H), 4.19 (s, 2H), 3.82 (s, 2H). Mass(m/z): 401.2[M+H]⁺

### 2-(3,5-dimethyl-1H-1,2,4-triazol-1-yl)-N-((7-(trifluoromethyl)-10H-phenoxazin-3-yl)methyl)acetamide (47)

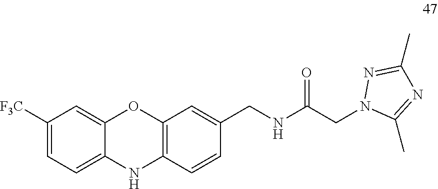 [SMILES: Cc1nc(C)n(CC(=O)NCc2ccc3c(c2)Oc2cc(C(F)(F)F)ccc2N3)n1]

The title compound 47 (3.6 mg) was prepared in a total yield of 8.6% as a yellow solid from (7-(trifluoromethyl)-10H-phenoxazin-3-yl)methanamine (28 mg, 0.1 mmol), 2-(3,5-dimethyl-1H-1,2,4-triazol-1-yl)acetic acid(15.5 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 6.97 (d, J=8.4 Hz, 1H), 6.75 (s, 1H), 6.69 (d, J=8.2 Hz, 1H), 6.58 (s, 1H), 6.46 (d, J=8.0 Hz, 1H), 6.39 (dd, J=8.0, 1.2 Hz, 1H), 4.82 (s, 2H), 4.19 (s, 2H), 2.40 (s, 3H), 2.27 (s, 3H). Mass(m/z): 418.2[M+H]⁺.

### N-((7-(trifluoromethyl)-10H-phenoxazin-3-yl)methyl)oxazole-4-carboxamide (48)

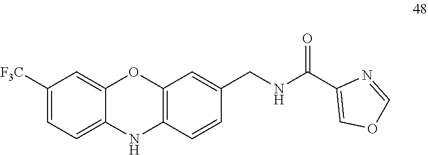 [SMILES: O=C(NCc1ccc2c(c1)Oc1cc(C(F)(F)F)ccc1N2)c1cocn1]

The title compound 48 (6.5 mg) was prepared in a total yield of 17.3% as a yellow solid from (7-(trifluoromethyl)-10H-phenoxazin-3-yl)methanamine (28 mg, 0.1 mmol), oxazole-4-carboxylic acid (11.3 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 8.40 (t, J=0.8 Hz, 1H), 8.19 (s, 1H), 6.99-6.94 (m, 1H), 6.75-6.71 (m, 2H), 6.60 (d, J=1.8 Hz, 1H), 6.45 (dd, J=8.2, 1.0 Hz, 1H), 6.39 (d, J=8.0 Hz, 1H), 4.33 (s, 2H). Mass(m/z): 376.1[M+H]+

### 2-(1H-pyrrol-2-yl)-N-((7-(trifluoromethyl)-10H-phenoxazin-3-yl)methyl)acetamide

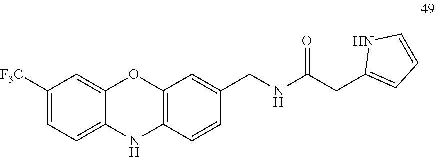 [SMILES: O=C(Cc1ccc[nH]1)NCc1ccc2c(c1)Oc1cc(C(F)(F)F)ccc1N2]

The title compound 49 (15.4 mg) was prepared in a total yield of 39.8% as a yellow solid from (7-(trifluoromethyl)-10H-phenoxazin-3-yl)methanamine (28 mg, 0.1 mmol), 2-(1H-pyrrol-2-yl)acetic acid (12.5 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ6.98-6.93 (m, 1H), 6.75-6.70 (m, 1H), 6.68-6.64 (m, 1H), 6.64-6.58 (m, 1H), 6.53-6.42 (m, 2H), 6.36-6.32 (m, 1H), 6.02 (t, J=3.2 Hz, 1H), 5.97-5.92 (m, 1H), 4.13 (s, 2H), 3.52 (s, 2H). Mass(m/z): 388.2[M+H]⁺

### 1-methyl-N-((7-(trifluoromethyl)-10H-phenoxazin-3-yl)methyl)-1H-1,2,4-triazole-5-carboxamide (50)

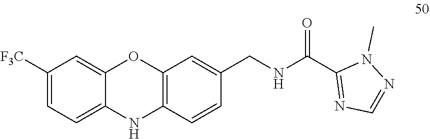 [SMILES: Cn1ncnc1C(=O)NCc1ccc2c(c1)Oc1cc(C(F)(F)F)ccc1N2]

The title compound 50 (6.5 mg) was prepared in a total yield of 16.7% as a yellow solid from (7-(trifluoromethyl)-10H-phenoxazin-3-yl)methanamine (28 mg, 0.1 mmol), 2-(1H-pyrrol-2-yl)acetic acid (12.7 mg, 0.1 mmol), DIEA (39 mg, 0.3 mmol) and HATU (38.0 mg, 0.1 mmol) according to the procedure for compound 4. ¹H NMR (400 MHz, Methanol-d₄) δ 7.90 (s, 1H), 6.99-6.94 (m, 1H), 6.76-6.72 (m, 2H), 6.63 (d, J=1.8 Hz, 1H), 6.46 (d, J=8.2 Hz, 1H), 6.40 (d, J=7.8 Hz, 1H), 4.33 (s, 2H), 4.21 (s, 3H). Mass(m/z): 390.2[M+H]⁺.

### 2-(4-ethylpiperazin-1-yl)-N-((7-(trifluoromethyl)-10H-phenoxazin-3-yl)methyl)acetamide (51)

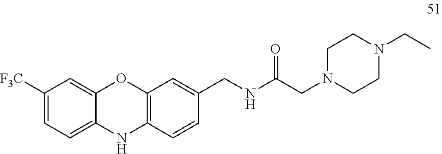 [SMILES: CCN1CCN(CC(=O)NCc2ccc3c(c2)Oc2cc(C(F)(F)F)ccc2N3)CC1]

The title compound 51 (5.3 mg) was prepared in a total yield of 12.2% as a yellow solid from 2-chloro-N-((7-(trifluoromethyl)-10H-phenoxazin-3-yl)methyl)acetamide (35.6 mg, 0.1 mmol) and 1-ethylpiperazine (114 mg, 1 mmol) according to the procedure for compound 34. ¹H NMR (400 MHz, Methanol-d₄) δ 6.99-6.94 (m, 1H), 6.74 (d, J=2.0 Hz, 1H), 6.69 (dd, J=8.0, 2.0 Hz, 1H), 6.57 (d, J=2.0 Hz, 1H), 6.47 (dd, J=8.2, 0.8 Hz, 1H), 6.39 (d, J=8.0 Hz, 1H), 4.21 (s, 2H), 3.58-3.43 (m, 2H), 3.21-2.96 (m, 8H), 2.71-2.51 (m, 2H), 1.34 (t, J=7.4 Hz, 3H).

Mass(m/z): 435.3[M+H]⁺.

### 2-(3,4-dimethylpiperazin-1-yl)-N-((7-(trifluoromethyl)-10H-phenoxazin-3-yl)methyl)acetamide (52)

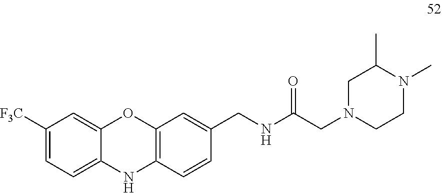 [SMILES: CC1CN(CC(=O)NCc2ccc3c(c2)Oc2cc(C(F)(F)F)ccc2N3)CCN1C]

The title compound 52 (7.2 mg) was prepared in a total yield of 16.6% as a yellow solid from 2-chloro-N-((7-(trifluoromethyl)-10H-phenoxazin-3-yl)methyl)acetamide (35.6 mg, 0.1 mmol) and 1,2-dimethylpiperazine (114 mg, 1 mmol) according to the procedure for compound 34. ¹H NMR (400 MHz, Methanol-d₄) δ 7.02-6.98 (m, 1H), 6.77 (d, J=2.0 Hz, 1H), 6.74-6.68 (m, 1H), 6.61-6.55 (m, 1H), 6.49 (d, J=8.2 Hz, 1H), 6.41 (dd, J=8.0, 1.0 Hz, 1H), 4.22 (s, 2H), 3.30-3.20 (m, 2H), 3.15 (s, 2H), 3.04-2.87 (m, 4H), 2.71 (s, 3H), 2.62-2.50 (m, 1H), 1.27 (d, J=6.4 Hz, 3H). Mass(m/z): 435.3[M+H]⁺.

### N-((7-(trifluoromethyl)-10H-phenoxazin-3-yl)methyl)-2-((3R,5S)-3,4,5-trimethylpiperazin-1-yl)acetamide (53)

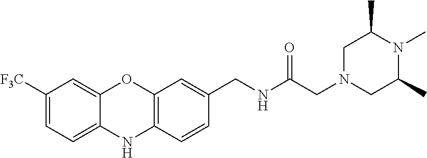 [SMILES: C[C@@H]1CN(CC(=O)NCc2ccc3c(c2)Oc2cc(C(F)(F)F)ccc2N3)C[C@H](C)N1C]

The title compound 53 (12.3 mg) was prepared in a total yield of 27.5% as a yellow solid from 2-chloro-N-((7-(trifluoromethyl)-10H-phenoxazin-3-yl)methyl)acetamide (35.6 mg, 0.1 mmol) and (2S,6R)-1,2,6-trimethylpiperazine (64 mg, 0.5 mmol) according to the procedure for compound 34. ¹H NMR (400 MHz, Methanol-d₄) δ 6.98 (d, J=8.2 Hz, 1H), 6.74 (s, 1H), 6.69 (dt, J=8.0, 1.6 Hz, 1H), 6.57 (t, J=1.6 Hz, 1H), 6.47 (d, J=8.2 Hz, 1H), 6.39 (dd, J=8.0, 1.2 Hz, 1H), 4.20 (s, 2H), 3.11 (s, 2H), 2.99-2.87 (m, 2H), 2.75-2.65 (m, 2H), 2.36-2.20 (m, 2H), 1.27 (d, J=6.8 Hz, 6H). Mass(m/z): 449.3[M+H]⁺.

### 2-(4-cyclopropylpiperazin-1-yl)-N-((7-(trifluoromethyl)-10H-phenoxazin-3-yl)methyl)acetamide (54)

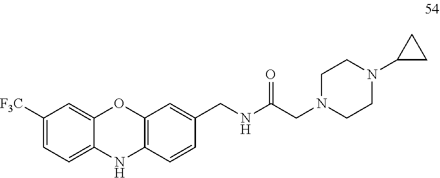 [SMILES: O=C(CN1CCN(C2CC2)CC1)NCc1ccc2c(c1)Oc1cc(C(F)(F)F)ccc1N2]

The title compound 54 (14.0 mg) was prepared in a total yield of 31.4% as a yellow solid from 2-chloro-N-((7-(trifluoromethyl)-10H-phenoxazin-3-yl)methyl)acetamide (35.6 mg, 0.1 mmol) and 1-cyclopropylpiperazine (63 mg, 0.5 mmol) according to the procedure for compound 34. ¹H NMR (400 MHz, Methanol-d₄) δ7.00-6.95 (m, 1H), 6.75 (d, J=2.0 Hz, 1H), 6.70-6.67 (m, 1H), 6.57 (d, J=2.0 Hz, 1H), 6.47-6.43 (m, 1H), 6.39 (d, J=8.0 Hz, 1H), 4.20 (s, 2H), 3.13 (s, 2H), 2.91-2.76 (m, 4H), 2.70-2.54 (m, 4H), 1.93-1.80 (m, 1H), 0.59-0.45 (m, 4H).

Mass(m/z): 447.3[M+H]⁺

### 2-((1S,4S)-5-methyl-2,5-diazabicyclo[2.2.1]heptan-2-yl)-N-((7-(trifluoromethyl)-10H-phenoxazin-3-yl)methyl)acetamide (55)

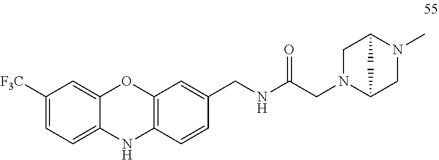 [SMILES: CN1C[C@@H]2C[C@H]1CN2CC(=O)NCc1ccc2c(c1)Oc1cc(C(F)(F)F)ccc1N2]

The title compound 55 (3.5 mg) was prepared in a total yield of 8.1% as a yellow solid from 2-chloro-N-((7-(trifluoromethyl)-10H-phenoxazin-3-yl)methyl)acetamide (35.6 mg, 0.1 mmol) (1S,4S)-2-methyl-2,5-diazabicyclo[2.2.1]heptane dihydrochloride (92 mg, 0.5 mmol) and DIEA (65 mg, 0.5 mmol) according to the procedure for compound 34. ¹H NMR (400 MHz, Methanol-d₄) δ6.99-6.57 (m, 1H), 6.74 (d, J=2.0 Hz, 1H), 6.70-6.67 (m, 1H), 6.56 (d, J=2.0 Hz, 1H), 6.47 (d, J=8.2 Hz, 1H), 6.38 (d, J=8.0 Hz, 1H), 4.21 (s, 2H), 4.13-4.10 (m, 1H), 3.67-3.63 (m, 1H), 3.36-3.31 (m, 4H), 3.00 (s, 2H), 2.90 (s, 3H), 2.20-2.07 (m, 2H). Mass(m/z): 433.3[M+H]⁺.

### N-((8-(trifluoromethyl)-10H-phenoxazin-3-yl)methyl)quinuclidine-4-carboxamide (56)

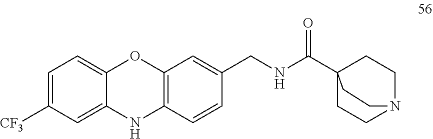 [SMILES: O=C(NCc1ccc2c(c1)Oc1ccc(C(F)(F)F)cc1N2)C12CCN(CC1)CC2]

The title compound 56 (16.9 mg) was prepared in a total yield of 31.8% as a yellow solid from (8-(trifluoromethyl)-10H-phenoxazin-3-yl)methanamine (28 mg, 0.1 mmol), 2-(1-methylpiperidin-4-yl)acetic acid (15.7 mg, 0.1 mmol), DIEA (39 mg, 0.3 mmol) and HATU (38.0 mg, 0.1 mmol) according to the procedure for compound 4. 1H NMR (400 MHz, Methanol-d₄) δ 6.83 (d, J=7.0 Hz, 1H), 6.65 (t, J=9.2 Hz, 2H), 6.60 (s, 1H), 6.54 (s, 1H), 6.36 (dt, J=7.8, 1.0 Hz, 1H), 4.18 (d, J=5.4 Hz, 2H), 3.44-3.36 (m, 6H), 2.15-2.08 (m, 6H).

Mass(m/z): 418.3 [M+H]⁺.

### 5-(dimethylamino)-N-((8-(trifluoromethyl)-10H-phenoxazin-3-yl)methyl)pentanamide (57)

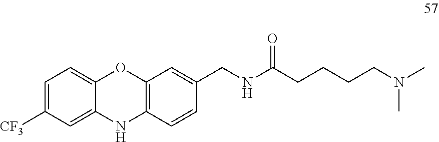 [SMILES: CN(C)CCCCC(=O)NCc1ccc2c(c1)Oc1ccc(C(F)(F)F)cc1N2]

The title compound 57 (2.6 mg) was prepared in a total yield of 6.6% as a yellow solid from (8-(trifluoromethyl)-10H-phenoxazin-3-yl)methanamine (28 mg, 0.1 mmol), 4-(dimethylamino)butanoic acid. hydrochloride salt (18 mg, 0.11 mmol), DIEA (39 mg, 0.3 mmol) and HATU (42 mg, 0.11 mmol) according to the procedure for compound 4. ¹H NMR (400 MHz, Methanol-d₄) δ 6.82 (d, J=8.2 Hz, 1H), 6.71-6.58 (m, 3H), 6.55 (s, 1H), 6.37 (d, J=8.0 Hz, 1H), 4.15 (s, 2H), 3.17-3.11 (m, 2H), 2.88 (s, 6H), 2.39 (t, J=6.8 Hz, 2H), 2.03-1.95 (m, 2H).

Mass(m/z): 394.2 [M+H]⁺

### N-((7-chloro-10H-phenoxazin-3-yl)methyl)-2-(4-methylpiperazin-1-yl)acetamide (58)

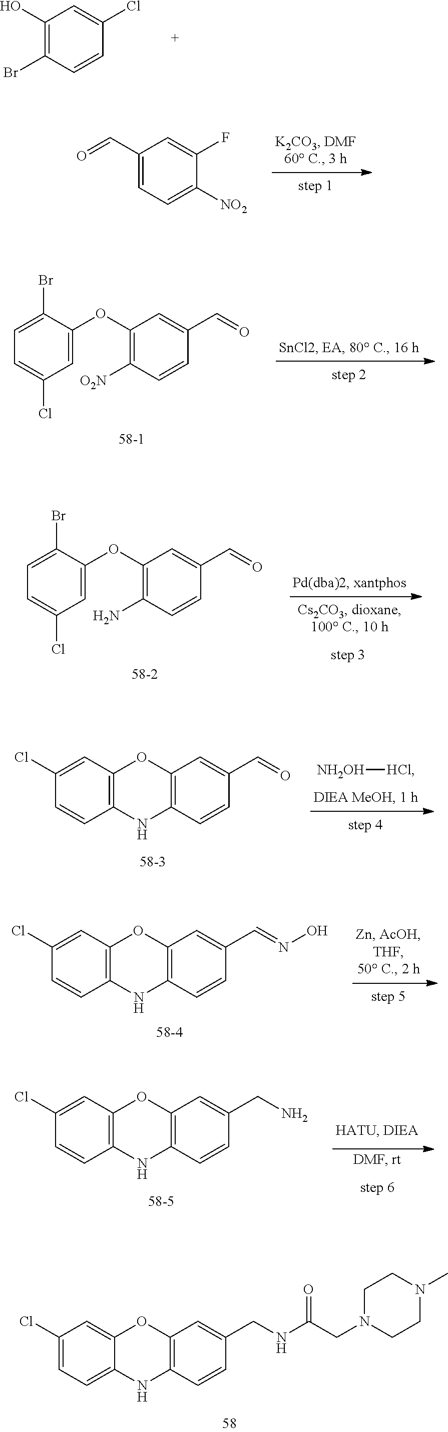

Step 1. A mixture of 2-bromo-5-chlorophenol (3 g, 0.01 mol), 3-fluoro-4-nitrobenzaldehyde (2 g, 0.012 mol), K₂CO₃ (2.7 g, 0.02 mol), DMF (50 mL) was stirred at 60° C. for 3 h. The mixture was concentrated and purified by flash, eluting with PE: EA=10:1 to 5:1 to give the desired product as yellow solid (4.26 g, 42.4%). Mass(m/z): 355.1 [M+H]⁺.

Step 2. A mixture of 3-(2-bromo-5-chlorophenoxy)-4-nitrobenzaldehyde (3 g, 8.4 mmol), SnCl₂(5.68 g, 25.2 mmol) and EA (30 mL) was stirred at 80° C. for 16 h. The mixture was basified with NaHCO₃ to pH=8, the mixture was poured into water, extracted with EA (20 mL×3). The mixture was concentrated and purified by flash eluting with PE: EA=10:1 to 5:1 to give the desired product as yellow solid (2.2 g, 14.05%). Mass(m/z): 326.3 [M+H]⁺.

Step 3. A mixture of 4-amino-3-(2-bromo-5-chlorophenoxy)benzaldehyde (1 g, 3.1 mmol), Pd₂(dba)₃ (283 mg, 0.31 mmol), Xantphos (269 mg, 0.46 mmol), Cs₂CO₃ (2.02 g, 6.2 mmol) and dioxane (20 mL) was stirred at 100° C. for 16 h. The mixture was concentrated and purified by flash, eluting with PE: EA=5:1 to 1:1 to give the desired product as yellow solid (630 mg, 91.64%). Mass(m/z): 246.6 [M+H]⁺.

Step 4. To a solution of 7-chloro-10H-phenoxazine-3-carbaldehyde (630 mg, 2.56 mmol), NH₂OH—HCl (266 mg, 3.84 mmol), DIEA (661 mg, 5.12 mmol) and MeOH (20 mL) was stirred at rt for 2 h. The mixture was poured into water, extracted with EA (20 mL×3), the organic layers were concentrated to give the desired product as yellow solid (550 mg, 31.26%).

Mass(m/z): 261.2 [M+H]⁺.

Step 5. A solution of (E)-7-chloro-10H-phenoxazine-3-carbaldehyde oxime (550 mg, 2.11 mmol), Zn (690 mg, 10.55 mmol) in THF/CH₃COOH=1:1 (20 mL) was stirred at 50° C. for 2 h. The mixture was basified with NaHCO₃ to pH=8, extracted with DCM/MeOH=10; 1, the organic layers were concentrated and purified by flash, eluting with DCM: MeOH (1% NH₃)=60:1 to 15:1 to give the desired product as yellow solid (103.5 mg, 59.7%). ¹H NMR (400 MHz, DMSO-d₆) δ 8.29 (s, 2H), 6.77 (s, 2H), 6.69 (s, 1H), 6.65 (s, 2H), 6.44-6.39 (m, 1H), 3.52 (s, 2H). Mass(m/z): 247.2 [M+H]⁺.

Step 6. The title compound 58 (9.8 mg) was prepared in a total yield of 25.4% as a yellow solid from (7-chloro-10H-phenoxazin-3-yl)methanamine (25 mg, 0.1 mmol), 2-(4-methylpiperazin-1-yl)acetic acid (16 mg, 0.1 mmol), DIEA (39 mg, 0.3 mmol) and HATU (38 mg, 0.1 mmol) according to the procedure for compound 4. ¹H NMR (400 MHz, Methanol-d₄) δ 6.74-6.67 (m, 2H), 6.60-6.54 (m, 2H), 6.40-6.33 (m, 2H), 4.22 (s, 2H), 3.46-3.33 (m, 6H), 3.06-2.88 (m, 7H). Mass(m/z): 387.2[M+H]⁺.

### N-((9-chloro-10H-phenoxazin-3-yl)methyl)-2-(4-methylpiperazin-1-yl)acetamide (59)

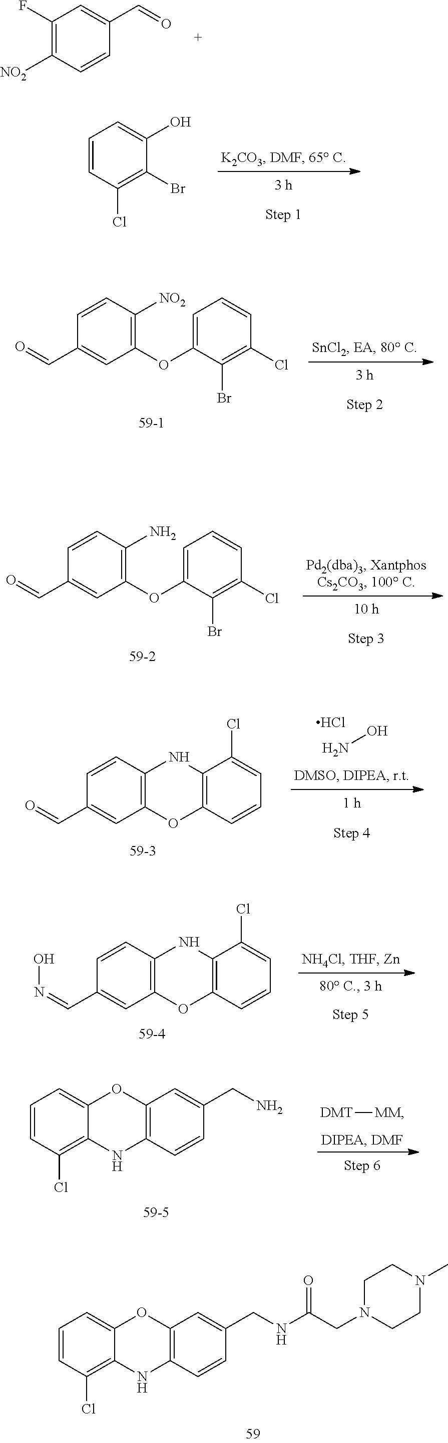

Step 1. A mixture of 3-fluoro-4-nitrobenzaldehyde (3 g, 17.7 mmol), 2-bromo-3-chlorophenol (4 g, 19.5 mmol), dipotassium carbonate (4.9 g, 35.4 mmol and DMF (15 mL) was stirred at 65° C. for 3 h. The mixture was diluted with EA (300 mL) and washed with water (300 mL×3). The organic phase was concentrated and purified by flash, eluting with PE: EA=10:1 to 1:1 to give the desired product as a yellow solid (2.7 g, 38.4%).

Step 2. To a solution of 3-(2-bromo-3-chlorophenoxy)-4-nitrobenzaldehyde (2.7 g, 7.6 mmol) and SnCl₂ (6.8 g, 30.4 mmol) in EA (30 mL) was stirred at 80° C. for 3 h. The mixture was diluted with EA (300 mL) and wash with water (150×2). The organic phase was concentrated and purified by flash, eluting with PE: EA=10:1 to 1:1 to give the desired product as a yellow solid (2 g, 72%). Mass(m/z): 326.0 [M+H]⁺.

Step 3. A mixture of 4-amino-3-(2-bromo-3-chlorophenoxy)benzaldehyde (1.9 g, 10 mmol), Pd2(dba)₃ (915 mg, 1 mmol), Xantphos (1.16 g, 2 mmol), Cs₂CO₃ (6.5 g, 20 mmol) and Tol (30 mL) was stirred at 100° C. under N₂ atmosphere for 10 h. The mixture was concentrated and purified by flash, eluting with PE: EA=10:1 to 1:1 to give the desired product as yellow solid (700 mg, 26%). Mass(m/z): 246.0 [M+H]⁺.

Step 4. To a solution of 9-chloro-10H-phenoxazine-3-carbaldehyde (500 mg, 2.8 mmol), hydroxylamine hydrochloride (396 mg, 5.7 mmol) and DIPEA (1.4 g, 11.4 mmol) in DMSO (5 mL) was stirred at 25° C. for 1 h. The mixture was diluted with water (100 mL) and filtrated, the solid was collected and dried to give the desired product as a yellow solid (600 mg, 72.7%).

Mass(m/z): 260.9 [M+H]⁺.

Step 5. To a solution of (Z)-9-chloro-10H-phenoxazine-3-carbaldehyde oxime (500 mg, 1.92 mmol) and Zn (1.2 g, 19.2 mmol) in NH₄Cl aq. (saturated, 10 mL) and THF (2 mL) was stirred at 80° C. for 3 h. The mixture was filtrated and extracted with THF (50 mL×2). The organic phase was concentrated and purified by flash, eluting with DCM: MeOH (10% NH3)=100:1 to 10:1 to give the desired product as a white solid (90.8 mg, 17.3%). ¹H NMR (400 MHz, DMSO-d₆) δ 7.84 (s, 1H), 6.86 (dd, J=7.1, 2.4 Hz, 1H), 6.77 (d, J=8.0 Hz, 1H), 6.70 (dd, J=8.0, 1.6 Hz, 1H), 6.65 (s, 1H), 6.60-6.55 (m, 2H), 3.51 (s, 2H). Mass(m/z): 230.1 [M−NH₂]⁺.

Step 6. The title compound 59 (29.4 mg) was prepared in a total yield of 62.3% as a white solid from (9-chloro-10H-phenoxazin-3-yl)methanamine (30 mg, 0.122 mmol) and 2-(4-methylpiperazin-1-yl)acetic acid (25 mg, 0.159 mmol) according to the procedure for compound 2. ¹H NMR (400 MHz, Methanol-d₄) δ 6.87-6.746.76 (m, 1H), 6.69-6.65 (m, 1H), 6.61 (dd, J=7.8, 0.8 Hz, 1H), 6.55 (dd, J=1.8, 0.8 Hz, 1H), 6.52 (dd, J=8.0, 0.8 Hz, 1H), 6.50-6.46 (m, 1H), 4.20 (s, 2H), 3.11 (s, 2H), 2.89 (s, 4H), 2.67 (s, 4H), 2.55 (s, 3H). Mass(m/z): 387.2 [M+H]⁺.

### 1-isopropyl-N-((7-(trifluoromethyl)-10H-phenoxazin-3-yl)methyl)piperidine-4-carboxamide (60)

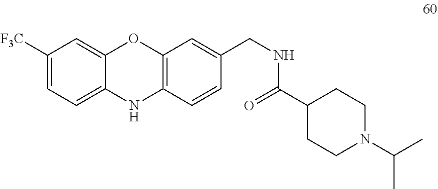 [SMILES: CC(C)N1CCC(C(=O)NCc2ccc3c(c2)Oc2cc(C(F)(F)F)ccc2N3)CC1]

The title compound 60 (5.9 mg) was prepared in a total yield of 13.3% as a yellow solid from (7-(trifluoromethyl)-10H-phenoxazin-3-yl)methanamine (28 mg, 0.1 mmol), isopropylpiperidine-4-carboxylic acid hydrochloride (22.8 mg, 0.11 mmol), DIEA (39 mg, 0.3 mmol) and HATU (45.6 mg, 0.12 mmol) according to the procedure for compound 4. ¹H NMR (400 MHz, Methanol-d₄) δ 6.99-6.96 (m, 1H), 6.74 (d, J=2.0 Hz, 1H), 6.67 (dd, J=8.0, 2.0 Hz, 1H), 6.55 (d, J=2.0 Hz, 1H), 6.47 (d, J=8.2 Hz, 1H), 6.39 (d, J=8.0 Hz, 1H), 4.15 (s, 2H), 3.53-3.46 (m, 3H), 3.12-2.99 (m, 2H), 2.61-2.51 (m, 1H), 2.12-1.99 (m, 4H), 1.35 (d, J=6.8 Hz, 6H). Mass(m/z): 434.3[M+H]⁺.

### 1-cyclopropyl-N-((7-(trifluoromethyl)-10H-phenoxazin-3-yl)methyl)piperidine-4-carboxamide (61)

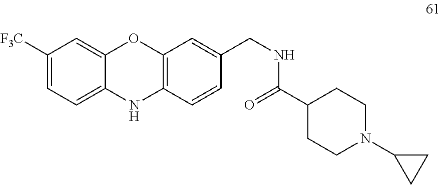 [SMILES: O=C(NCc1ccc2c(c1)Oc1cc(C(F)(F)F)ccc1N2)C1CCN(C2CC2)CC1]

The title compound 61 (7.1 mg) was prepared in a total yield of 23.1% as a yellow solid from (7-(trifluoromethyl)-10H-phenoxazin-3-yl)methanamine (20 mg, 0.07 mmol), 1-cyclopropylpiperidine-4-carboxylic acid (13 mg, 0.08 mmol), DIEA (28 mg, 0.21 mmol) and HATU (33 mg, 0.09 mmol) according to the procedure for compound 4. ¹H NMR (400 MHz, Methanol-d₄) δ 7.00-6.95 (m, 1H), 6.74 (d, J=2.0 Hz, 1H), 6.69-6.65 (m, 1H), 6.54 (d, J=1.8 Hz, 1H), 6.47 (d, J=8.2, 1H), 6.38 (d, J=8.0 Hz, 1H), 4.15 (s, 2H), 3.69-3.60 (m, 2H), 3.26-3.11 (m, 2H), 2.83-2.72 (m, 1H), 2.61-2.51 (m, 1H), 2.10-1.88 (m, 4H), 1.00-0.92 (m, 4H).

Mass(m/z): 432.3[M+H]⁺

### 1-methyl-2-oxo-N-((8-(trifluoromethyl)-10H-phenoxazin-3-yl)methyl)piperidine-4-carboxamide (62)

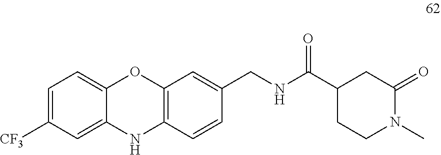 [SMILES: CN1CCC(C(=O)NCc2ccc3c(c2)Oc2ccc(C(F)(F)F)cc2N3)CC1=O]

The title compound 62 (21.0 mg) was prepared in a total yield of 46.7% as a white solid from (8-(trifluoromethyl)-10H-phenoxazin-3-yl)methanamine (30 mg, 0.107 mmol) and 1-methyl-2-oxopiperidine-4-carboxylic acid (17 mg, 0.107 mmol) according to the procedure for 4.

¹H NMR (400 MHz, Methanol-d₄) δ 6.81 (ddt, J=8.2, 2.0, 0.8 Hz, 1H), 6.68-6.59 (m, 3H), 6.53 (d, J=2.0 Hz, 1H), 6.38 (d, J=8.0 Hz, 1H), 4.14 (d, J=2.8 Hz, 2H), 3.40-3.34 (m, 2H), 2.92 (s, 3H), 2.78 (dtd, J=10.6, 5.8, 3.0 Hz, 1H), 2.59-2.43 (m, 2H), 2.08-2.01 (m, 1H), 1.92 (dddd, J=13.4, 10.4, 9.0, 6.4 Hz, 1H). Mass(m/z): 420.3[M+H]⁺.

### N-((10H-phenothiazin-3-yl)methyl)-2-(4-methylpiperazin-1-yl)acetamide (63)

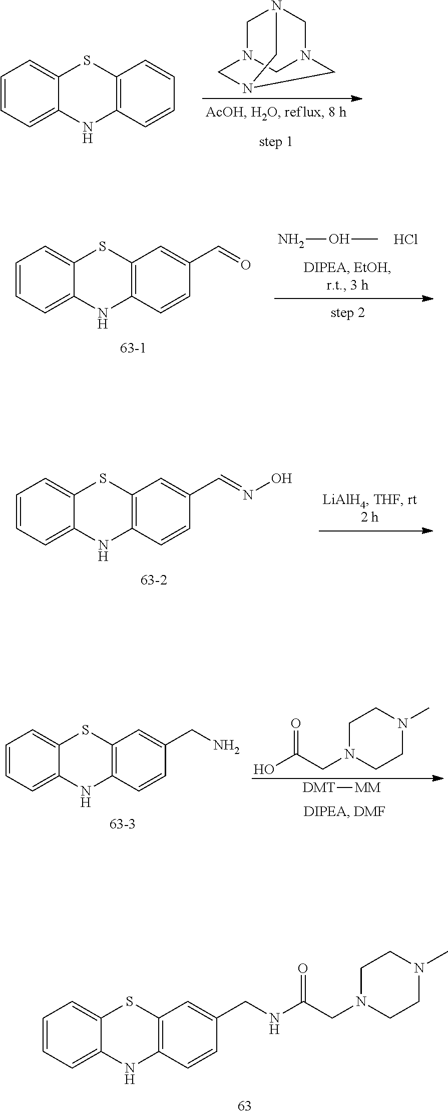

### Preparation of 10H-phenothiazine-3-carbaldehyde

### Step 1. Preparation of 10H-phenothiazine-3-carbaldehyde

A mixture of 10H-phenothiazine (5 g, 25.09 mmol), (1s,3s,5s)-1,3,5,7-tetraazaadamantane (10.55 g, 75.27 mmol), AcOH (120 mL) and H₂O (60 mL) was stirred at 120° C. for 2 h. The mixture was concentrated and purified by flash, eluting with PE: EA=3:1 to 1:1 to give the desired product as yellow solid (2.5 g, 43.8%).

### Step 2. Preparation of (E)-10H-phenothiazine-3-carbaldehyde oxime

To a solution of 10H-phenothiazine-3-carbaldehyde (2.5 g, 11.00 mmol) in EtOH (50 mL) was added NH2-OH— HCl (1.53 g, 22.00 mmol) and DIPEA (4.26 g, 33.00 mmol), then the mixture was stirred at 25° C. for 3 h and water (50 mL) was added into the mixture, then filtered to afford the product. Mass(m/z): 242.9 [M+H]⁺.

### Step 3. Preparation of (10H-phenothiazin-3-yl)methanamine (63-3)

A mixture of (E)-10H-phenothiazine-3-carbaldehyde oxime (400 mg, 1.65 mmol) and THF (5 mL) was added LiAlH₄ (187.9 mg, 4.95 mmol) and stirred at 25° C. for 2 h. The mixture was quenched by water and 15% NaOH, filtered and dried over Na₂SO₄, concentrated and purified by flash eluting with DCM:MeOH=1:0 to 5:1 to give the desired product as white solid (30 mg, 7.96%). ¹H NMR (300 MHz, DMSO-d₆) δ 8.49 (s, 1H), 6.98-6.78 (m, 4H), 6.74-6.56 (m, 3H), 3.56 (s, 2H). Mass(m/z): 212.1 [M−NH₂]⁺.

Step 4. The title compound 63 (9.3 mg) was prepared in a total yield of 19.2% as a white solid from (10H-phenothiazin-3-yl)methanamine (30 mg, 0.132 mmol) and 2-(4-methylpiperazin-1-yl)acetic acid (27 mg, 0.171 mmol). ¹H NMR (400 MHz, Methanol-d₄) δ 6.97-6.93 (m, 1H), 6.91 (dd, J=5.4, 1.8 Hz, 1H), 6.87 (dd, J=10.8, 1.8 Hz, 1H), 6.84 (t, J=1.3 Hz, 1H), 6.73 (td, J=7.6, 1.4 Hz, 1H), 6.61 (dd, J=7.8, 1.2 Hz, 1H), 6.58 (d, J=8.0 Hz, 1H), 4.23 (s, 2H), 3.14 (s, 3H), 3.05 (m, 4H), 2.72 (m, 4H), 2.67 (s, 3H). Mass(m/z): 369.3 [M+H]⁺.

1. A compound of formula I, or a salt, hydrate or stereoisomer thereof: 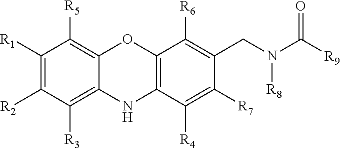
wherein:
R1-R8 are independently H, optionally substituted heteroatom or optionally substituted hydrocarbyl; and
R9 is optionally substituted alkyl or optionally substituted, optionally heterocyclic.

2. The compound of claim 1 wherein:
R1-R7 are independently H, optionally substituted heteroatom or optionally substituted hydrocarbyl;
R1-R7 are independently H, halide, optionally substituted OH or NH2, or optionally substituted alkyl;
R1-R7 are independently H or optionally F-substituted lower alkyl;
R8 is H, optionally substituted heteroatom or optionally substituted hydrocarbyl;
R8 is H, optionally substituted OH or optionally substituted alkyl;
R8 is H or OH;
R9 is optionally substituted alkyl or optionally substituted, optionally hetero cyclic;
R9 is substituted methyl;
R9 is methyl substituted with optionally substituted optionally heterocyclic;
1, 2, 3, 4 or 5 of R3-R7 are H; or
any combination of the foregoing substituents.

3. The compound of claim 1 having a structure disclosed herein.

4. A pharmaceutical composition comprising a therapeutically effective amount of a compound of any claim herein and one or more pharmaceutically acceptable excipients, in predetermined, unit dosage form.

5. Use of a compound or composition of any of claim herein in the manufacture of a medicament to inhibit ferroptosis activity, or modulate or inhibit a disease associated with ferroptosis dysregulation, such as neuropathy, ischemia reperfusion injury, acute kidney failure and cancer, in a person in need thereof.

6. A compound or composition of any claim herein to inhibit ferroptosis activity, or modulate or inhibit a disease associated with ferroptosis dysregulation, such as neuropathy, ischemia reperfusion injury, acute kidney failure and cancer, in a person in need thereof, or in the manufacture of a medicament thereof in a person in need thereof.

7. A method of using a compound or composition of any claim herein to inhibit ferroptosis activity, or modulate or inhibit a disease associated with ferroptosis dysregulation, such as neuropathy, ischemia reperfusion injury, acute kidney failure and cancer, and optionally detecting a resultant improvement in the person's health or condition.
